(19) United States
(12) Patent Application Publication
(10) Pub. No.: US 2004/0028756 A1
(43) Pub. Date: Feb. 12, 2004

(54) Platelet-activated factor antagonists as analgesic, anti-inflammatory, uterine contraction inhibiting, and anti-tumor agents

(76) Inventors: Lisa A. Teather, St. Catharines (CA); Richard J. Wurtman, Boston, MA (US); Jane E. Magnusson, Auckland (NZ)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/397,228

(22) Filed: Mar. 27, 2003

Related U.S. Application Data
(60) Provisional application No. 60/367,488, filed on Mar. 27, 2002.
(60) Provisional application No. 60/367,489, filed on Mar. 27, 2002.

(51) Int. Cl. A61K 35/78; A61K 31/5513; A61K 31/34

(52) U.S. Cl. 424/752; 514/221; 514/473; 424/756

(57) ABSTRACT

Antagonists to platelet-activating factor provide analgesic effects as well as limit the release of inflammatory mediators. Use of these antagonists in the form of pharmaceutical compositions or nutritionals is beneficial (1) in the treatment of acute and/or chronic pain; (2) in the inhibition of inappropriate or excessive contraction of the uterus; (3) in the treatment of septic shock; and (4) in the inhibition of angiogenesis and/or tumor cell proliferation.

### CROSS REFERENCE TO RELATED APPLICATIONS

This application claims the benefit of priority under 35 U.S.C. § 119(e) based on copending U.S. Provisional Application No. 60/367,488, filed Mar. 27, 2002 and No. 60/367,489, filed Mar. 27, 2002, the disclosures of each of which are herein incorporated by reference in their entirety.

### STATEMENT REGARDING FEDERALLY SPONSORED RESEARCH OR DEVELOPMENT

The invention described herein was supported in whole or in part by grants from The National Institutes of Mental Health (Grant No. 5-RO1 MH28783-24) and The Center for Brain Sciences and Metabolism Charitable Trust.

### FIELD OF THE INVENTION

This invention relates generally to beneficial effects obtained via administration of antagonists to platelet-activating factor. In particular, this invention relates to treatment of acute or chronic pain, inhibition of inappropriate or excessive contraction of the uterus, treatment of septic shock, and inhibition of angiogenesis.

### BACKGROUND OF THE INVENTION

Platelet Activating Factor, or PAF (1-O-alkyl-2-acetyl-sn-glycero-3-phosphocholine) is a family of structurally related and biologically potent phospholipid mediators. PAF is a membrane-derived mediator that has biological effects on a variety of cells and tissues. A variety of stimuli, including those producing inflammation, promote the synthesis and release of PAF from various cell types.

PAF is synthesized in and released by various cells in the PNS and CNS. PAF subsequently activates surrounding cells, such as glial and endothelial cells, (M. S. Aihara, et al., Interaction between neuron and microglia mediated by platelet-activating factor, Genes to Cells 5 (2000) 397-406). PAF also mediates the expression of the inducible cyclooxygenase (COX) enzyme, cyclooxygenase-2 (COX-2), (N. G. Bazan, et al., Platelet-activating factor and retinoic acid synergistically activate the inducible prostaglandin synthase gene, Proc. Natl. Acad. Sci. USA. 91 (1994) 52525256), an enzyme responsible for the initial steps in the conversion of free arachidonic acid (AA) to prostaglandins (PGs) during inflammation, (for a review, see J. R. Vane, et al., Cyclooxygenases 1 and 2, Ann. Rev. Pharm. Toxicol. 38 (1998) 97-120).

PAF exerts cellular actions through high affinity intracellular membrane-binding sites, as well as through low-affinity cell surface receptors, (Marcheselli et al., Distinct platelet-activating factor binding sites in synaptic endings and in intracellular membranes of rat cerebral cortex, J. Biol. Chem. 265 (1990) 9140-9145). The binding of PAF to cell surface receptors results in the activation of diverse intracellular signal transduction pathways that ultimately activate transcription factors and induce gene expression. For example, calcium, cyclic AMP (cAMP), inositol 1,4,5-triphosphate (IP₃), and diacylglycerol (DAG) can function as second messengers for signaling by the plasma membrane PAF receptor, (for review, see Ishii & Shimizu, Platelet-activating factor (PAF) receptor and genetically engineered PAF receptor mutant mice. Prog Lipid Res 39 (2000) 41-82). Moreover, PAF also acts as an intracellular mediator, (Marcheselli et al., Platelet-activating factor is a messenger in the electroconvulsive shock-induced transcriptional activation of c-fos and zif-268 in hippocampus. J Neurosci Research 37 (1994) 54-61; Bazan et al., Platelet-activating factor and intracellular signaling pathways that modulate gene expression. In: Platelet-Activating Factor Receptors: Signal Mechanisms and Molecular Biology (ed. S. Shukla), (1993) pp. (137-146). CRC Press Inc., Boca Raton), binding to intracellular sites, which then elicit gene expression in neuronal and glial cell lines.

While early animal studies relating to PAF antagonists were encouraging, more recent studies, (see for example Heller et al, 1998) have been disappointing. Thus, The Pharmacologic Basis of Therapeutics states “[ . . . ] it appears as though currently available PAF antagonists are of little benefit in human disease.” (Goodman & Gilman, The Pharmacologic Basis of Therapeutics 10^{th} edition, 2001, edited by J. Hardman and L. Limbird, p. 682.) The reason for such discouraging results in previous work may be due to the existence of distinct sites of PAF interaction. PAF antagonists appear to be more selective for one site or the other. Thus, certain pathological effects of PAF may be caused by intercellular site actions making most of the cell surfact selective inhibitors unsuccessful

### SUMMARY OF THE INVENTION

The present invention relates to methods of controlling or alleviating pain by controlling the activation of astrocytes and/or other cell types and thus preventing these cells from releasing harmful substances that kill or overexcite surrounding neurons.

The present invention also relates to the use of PAF antagonists that act preferentially at the cell surface site in diseases involving excitotoxicity; such as ischemia and stroke.

The present invention also relates to the use of PAF antagonists that act preferentially at the intracellular binding sites in inflammatory/immune-based disorders, such as sepsis, alzheimer's, ALS, multiple sclerosis.

The present invention also relates to the combined use of PAF antagonists having different selectivity in those diseases or disorders where PAF is having pathological effect at both surface and intracellular sites.

In one aspect of the present invention, a method is provided for the use of drugs or nutritionals to diminish pain or inflammation comprising blocking one or more receptors for platelet-activating factor.

In another aspect of the present invention, a method is provided for the use of drugs or nutritionals to diminish pain or inflammation comprising blocking one or more cell surface receptors for platelet-activating factor and/or by blocking one or more intracellular receptor binding sites for platelet-activating factor.

In another aspect of the present invention, a method is provided for the use of nutritionals related to Gingko biloba and its constituents to diminish pain or inflammation comprising blocking one or more receptors for platelet-activating factor.

In another aspect of the present invention, a method is provided for the use of synthetic drugs related to benzodiazapines to diminish pain or inflammation comprising blocking one or more receptors for platelet-activating factor.

In another aspect of the present invention, a method is provided for the use of synthetic drugs related to tetrahydrofurans to diminish pain or inflammation comprising blocking one or more receptors for platelet-activating factor.

In another aspect of the present invention, a method is provided for the use of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl)hexanolamine, octylonium bromide, PCA-4248, and tetrahydrocannabinol-7-oic acid, either alone or in combination, to diminish pain or inflammation comprising blocking one or more receptors for platelet-activating factor.

In another aspect of the present invention, a method is provided for the use of compounds that inhibit prostaglandin synthesis by decreasing or abolishing platelet-activating factor actions to treat pain.

In another aspect of the present invention, a method is provided for the use of drugs or nutritionals to inhibit the inappropriate or excessive contraction of the uterus comprising blocking one or more receptors for platelet-activating factor.

In another aspect of the present invention, a method is provided for inhibiting pain and/or cramps associated with premenstrual syndrome (also known as late luteal phase dysphoric disorder, or premenstrual dysphoric disorder) comprising blocking one or more receptors for platelet-activating factor.

In another aspect of the present invention, a method is provided for inhibiting pain and/or cramps associated with normal menses comprising blocking one or more receptors for platelet-activating factor.

In another aspect of the present invention, a method is provided for inhibiting spontaneous abortion/miscarriage comprising blocking one or more receptors for platelet-activating factor.

In another aspect of the present invention, a method is provided for inhibiting pain, cramping, and/or discomfort associated with the perimenopausal period comprising blocking one or more receptors for platelet-activating factor.

In another aspect of the present invention, a method is provided for reducing pain associated with childbirth, including pain experienced during and post labor comprising blocking one or more receptors for platelet-activating factor.

In another aspect of the present invention, a method is provided for inhibiting Braxton Hicks contractions comprising blocking one or more receptors for platelet-activating factor.

In another aspect of the present invention, a method is provided for inhibiting the initiation and/or the severity of septic shock comprising one or more blocking receptors for platelet-activating factor.

In another aspect of the present invention, a method is provided for inhibiting the proliferation of tumor cells comprising blocking receptors for platelet-activating factor.

In another aspect of the present invention, a method is provided for inhibiting neo-angiogenesis comprising blocking one or more receptors for platelet-activating factor.

The above-recited methods are accomplished by the administration of a therapeutically effective amount of one or more antagonists to platelet activating factor that either block one or more receptors for platelet-activating factors or block one or more binding sites for platelet-activating factors. These antagonists can be pharmaceuticals or nutraceuticals.

For each of the above-recited methods of the present invention, the therapeutically effective amount of one or more PAF antagonists may be administered in conjunction with a therapeutically effective amount of one or more anti-inflammatory compounds and/or a therapeutically effective amount of one or more immunomodulatory agents.

In certain embodiments of the method of the present invention, the antiinflammatory compound or immunomodulatory drug comprises interferon; interferon derivatives comprising betaseron, .beta.-interferon; prostane derivatives comprising iloprost, cicaprost; glucocorticoids comprising cortisol, prednisolone, methyl-prednisolone, dexamethasone; immunsuppressives comprising cyclosporine A, FK506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in combination.

In one aspect of the invention, the therapeutically effective amount of the one or more antagonists to platelet activating factor administered is that amount sufficient to reduce or inhibit, inter alia, the pain associated with one or more of the following diseases: ischemia, stroke, sepsis, amyotrophic lateral sclerosis (ALS), epilepsy, extension of strokes after initial tissue damage, Alzheimer's disease, Parkinson's disease, Huntington's disease, functional brain damage secondary to primary and secondary brain tumors, Pick's disease, diffuse Lewy body disease, progressive supranuclear palsy, cerebellar degeneration, Shy-drager syndrome, amyotrophic lateral sclerosis, spinal muscular atrophy, multiple sclerosis, local brain damage secondary to meningitis or brain abscess, viral meningitis, viral encephalitis, HIV neurological disease, and/or local brain damage secondary to trauma.

In another aspect of the invention, the therapeutically effective amount of the one or more antagonists to platelet activating factor administered is that amount sufficient to inhibit the inappropriate or excessive contraction of the uterus, inhibit the pain and/or cramps associated with premenstrual syndrome (also known as late luteal phase dysphoric disorder, or premenstrual dysphoric disorder), inhibit the pain and/or cramps associated with normal menses, inhibit spontaneous abortion/miscarriage, inhibit the pain, cramping, and/or discomfort associated with the perimenopausal period, reduce the pain associated with childbirth, including pain experienced during and post labor, inhibit Braxton Hicks contractions, inhibit the initiation and/or the severity of septic shock, inhibit the proliferation of tumor cells, and/or inhibit neo-angiogenesis.

In one embodiment, the reduction or inhibition of pain and/or symptoms associated with one or more of each of the above-recited indications is on the order of about 10-20% reduction or inhibition. In another embodiment, the reduction or inhibition of pain is on the order of 30-40%. In another embodiment, the reduction or inhibition of pain is on the order of 50-60%. In yet another embodiment, the reduction or inhibition of the pain associated with each of the recited indications is on the order of 75-100%. It is intended herein that the ranges recited also include all those specific percentage amounts between the recited range. For example, the range of about 75 to 100% also encompasses 76 to 99%, 77 to 98%, etc, without actually reciting each specific range therewith.

In yet another aspect, the present invention is directed to a method of relieving or ameliorating the pain or symptoms associated with any one or more of the above-identified diseases or indications in a mammal suffering from any one or more of the above-identified diseases or indications which comprises administering to the mammal in need thereof a therapeutically effective pain or symptom-reducing amount of a pharmaceutical composition comprising one or more antagonists to platelet activating factor, either alone or in combination with one or more anti-inflammatory compounds or immunomodulatory agents; and a pharmaceutically acceptable carrier or excipient.

In one aspect of the invention, the one or more one or more antagonists to platelet activating factor of the present invention are administered orally, systemically, via an implant, intravenously, topically, or intrathecally.

In certain embodiments of the methods of the present invention, the subject or mammal is a human.

In other embodiments of the methods of the present invention, the subject or mammal is a veterinary and/or a domesticated mammal.

There has been thus outlined, rather broadly, the important features of the invention in order that a detailed description thereof that follows can be better understood, and in order that the present contribution can be better appreciated. There are additional features of the invention that will be described hereinafter.

In this respect, before explaining at least one embodiment of the invention in detail, it is to be understood that the invention is not limited in its application to the details as set forth in the following description and figures. The present invention is capable of other embodiments and of being practiced and carried out in various ways. Additionally, it is to be understood that the terminology and phraseology employed herein are for the purpose of description and should not be regarded as limiting.

As such, those skilled in the art will appreciate that the conception, upon which this disclosure is based, can readily be used as a basis for designing other methods for carrying out the several features and advantages of the present invention. It is important, therefore, that the claims be regarded as including such equivalent constructions insofar as they do not depart from the spirit and scope of the present invention.

### BRIEF DESCRIPTION OF THE DRAWINGS

FIG. 1 illustrates time-dependent PGE₂ release induced by mc-PAF from astrocyte-enriched cortical cell cultures. Cells are incubated with 1 μM mc-PAF or vehicle (0.01% methanol) at 37° C. for various times. Media is collected and assayed for PGE₂ (as described in materials and methods). Each point represents the mean +/− SEM of at least 3 independent experiments, carried out in duplicate or triplicate. *indicates statistically significant (p<0.05) differences relative to control.

FIGS. 2A and B illustrate PGE₂ release from astrocyte-enriched cortical cell cultures exposed to (FIG. 2A) mc-PAF, lyso-PAF, PAF-16, or PAF-18 and (FIG. 2B) mc-PAF, lyso-PC or PC. Cells are incubated in the respective treatments at 37° C. for 30 min, at which time the media is collected and assayed for PGE₂ (as described in materials and methods). Each point represents the mean +/− SEM of at least 3 independent experiments, carried out in duplicate or triplicate. The mean +/− SEM for control cultures was 35.6+/−7.9 and significant differences are indicated in results section.

FIGS. 3A, B, and C illustrate concentration-dependent PGE₂ release from media of astrocyte-enriched cortical cell cultures exposed to (FIG. 3A) AA (0.0110 μM) and (FIG. 3B) AA (0.01 μM) with or without mc-PAF (0.01-1 μM) and (FIG. 3C) AA (10 μM) with or without mc-PAF (0.01-1 μM). Cells are incubated in various concentrations of AA (with or without mc-PAF) or vehicle (0.01% ethanol, 0.01% methanol or both) at 37° C. for 30 min, at which time the media is collected and assayed for PGE₂ (as described in materials and methods). Each point represents the mean +/− SEM of at least 3 independent experiments, carried out in duplicate or triplicate. * indicates statistically significant (p<0.05) differences relative to control and ** relative to AA alone.

FIGS. 4A, B, and C illustrate the PAF antagonist, BN 50730 attenuates the (FIG. 4A) mc-PAF-, (FIG. 4B) lyso-PAF- and (FIG. 4C) AA-induced PGE₂ release in astrocytes in concentration-dependent manners. Cells are incubated at 37° C. for 30 min with various concentrations of BN 50730 before addition of mc-PAF (1 μM). After 30 min, media is collected and assayed for PGE₂ (as described in materials and methods). Each point represents the mean +/− SEM of at least 3 independent experiments, carried out in duplicate or triplicate. * indicates statistically significant (p<0.05) differences relative to control and ** relative to mc-PAF, lyso-PAF or AA alone.

FIGS. 5A and B illustrate the PAF antagonists, (FIG. 5A) BN 52021 (150 μM) and (FIG. 5B) CV 6209 (1-50 μM) do not attenuate the mc-PAF-induced PGE₂ release in astrocytes in concentration-dependent manners. Cells are incubated at 37° C. for 30 min in the respective antagonists before addition of mc-PAF (1 μM). After 30 min, media was collected and assayed for PGE₂ (as described in materials and methods). Each point represents the mean +/− SEM of at least 3 independent experiments, carried out in duplicate or triplicate. * indicates statistically significant (p<0.05) differences relative to control.

FIGS. 6A and B illustrate formalin-evoked nociceptive responses in rats that receive systemic BN 52021 (10, 1 or 0.1 mg/kg) or control injections. Total paw elevation times in (FIG. 6A) the early phase (0-10 min after injection) and (FIG. 6B) the late phase (10-60 min after injection) of formalin-induced nociception. Data are expressed as means +/− SEM. * p<0.05; Fisher's PLSD test vs control. HBC 45% hydroxypropyl-B-cyclodextrin (in distilled water).

FIGS. 7A and B illustrate formalin-evoked nociceptive responses in rats that received systemic BN 50730 (10, 1 or 0.1 mg/kg) or control injections. Total paw elevation times in (FIG. 7A) the early phase (0-10 min after injection) and (FIG. 7B) the late phase (10-60 min after injection) of formalin-induced nociception. Data are expressed as means =/− SEM. * p<0.05; Fisher's PLSD test vs control. HBC=45% hydroxypropyl-B-cyclodextrin (in distilled water).

FIG. 8 illustrates the effect of PGE₂ release from primary cortical astrocytes exposed to the non-hydrolyzable analog of PAF, methylcarbamyl-PAF (mc-PAF). Cells were incubated at 37° C. with various mc-PAF concentrations for 30 min, at which time the media was collected and assayed for PGE_{2.} Each point represents the mean +/−S.E.M. of at least three independent experiments, carried out in triplicate. The mean mean +/−S.E.M. for control cultures was 0.8+\−0.011.

FIGS. 9A and 9B illustrate that preferential COX-1-selective inhibitors have minimal influence on the mc-PAF-induced PGE₂ release from astrocytes. Cells were incubated at 37° C. with various concentrations of (A) piroxicam or (B) SC-560 for 30 min prior to addition of mc-PAF (0.1 μM) for 30 min, at which time the media was collected and assayed for PGE_{2.} Each point represents the mean +/−S.E.M. of at least three independent experiments, carried out in triplicate. *, Statistically significant (P<0.05) difference relative to control and **, relative to mc-PAF.

FIGS. 10A and 10B illustrate that inhibition of COX-2 attenuates mc-PAF-induced PGE₂ release from astrocytes. Cells were incubated at 37° C. with various concentrations of (A) the noselective COX inhibitor indomethacin or (B) the COX-2 selective inhibitor NS-398 for 30 min prior to addition of mc-PAF (0.1 μM) for 30 min, at which time the media was collected and assayed for PGE_{2.} Each point represents the mean +/−S.E.M. of at least three independent experiments, carried out in triplicate. *, Statistically significant (P<0.05) difference relative to control and **, relative to mc-PAF.

### DETAILED DESCRIPTION OF THE INVENTION

Prostaglandins (PGs) have important functions in brain cells, and mediate a variety of neuropathologic phenomena, including such inflammation-associated disorders as Alzheimer's disease (AD) and amyotrophic lateral sclerosis (ALS). When cells and tissue are exposed to various stimuli, arachidonic acid (AA) is liberated from membrane phospholipids and is converted to prostanoids, including PGs, by the action of cyclooxygenase (COX) enzymes. Two related but unique isoforms of COX, cyclooxygenase-1 (COX-1) and cyclooxygenase-2 (COX-2) catalyze identical reactions, a cyclooxygenation to form PGG2, and a peroxidation which reduces PGG2 to PGH2, the precursor of all other PGs, including PGE_{2.} COX-1 is constitutively expressed by most cells and is considered to be involved in maintaining cell homeostasis; in contrast the mitogen-inducible COX-2 is implicated in inflammatory and immune responses.

Astrocytes have an important role in CNS inflammation/immune responses. Following CNS injury or an immune/inflammatory challenge, astrocytes undergo a phenotypic alteration—a response known as activation. The activated astrocytes then release cytokines and other pro-inflammatory mediators, including PGs. These released substances communicate with (and ultimately affect the function of) such neighboring cells as neurons and microvascular cells. Astrocytes are a major source of PGs in the CNS; in culture these cells synthesize up to 20 times more PGs than do neurons. PGE₂ is the major AA metabolite involved in modulation of immunoinflammatory responses.

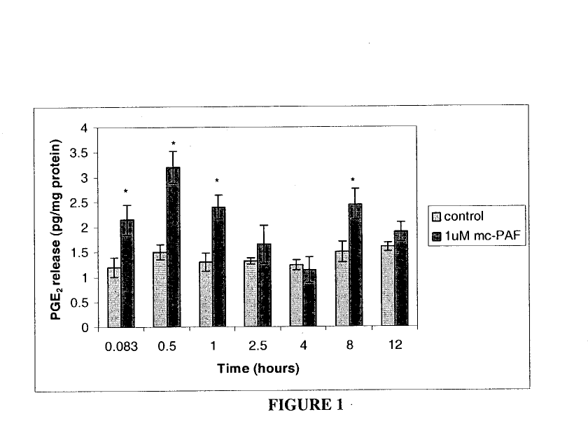

The acute or immediate phase of inflammation is the earliest response to tissue injury, as well as to immunological or pro-inflammatory challenges. COX-1 is often responsible for the immediate increases in PGs produced by various types of inflammatory stimuli, and COX-2 for the increased levels characteristic of the delayed phase of inflammation. However, the degree to which each COX isoform contributes to particular acute inflammatory responses depends upon such factors as the nature of the inflammatory stimulus and the cell type involved. As disclosed in copending patent applications (Patent Application Nos. 60/367,488 and 60/367,489), the pro-inflammatory mediator PAF, acting at micosomal binding sites, increases the release of PGE₂ from cortical astrocytes. This effect is observed within minutes of PAF stimulation and PGE₂ accumulation peaks at 30 minutes, suggesting that PAF induces an acute inflammatory reaction in astrocytes. As FIG. 1 shows, PAF increases PGE₂ release at 8 hours. Therefore, PAF may also have a role I delayed inflammatory reactions as well.

The experimental results set forth herein, describe a role for endogenous PAF in nociceptive transmission, especially for persistent pain. The findings also indicate that both intracellular and cell surface PAF binding sites are involved in nociceptive modulation in rats, and that PAF antagonists are useful for treating patients having acute or chronic pain. As described herein, the nociceptive responses to subcutaneous formalin injection are significantly reduced in rats receiving PAF antagonists that act on intracellular or cell surface PAF binding sites. In a preferred embodiment, treatment comprises administration of at least two PAF antagonists, each having a different selectivity. One site may be sufficient for treatment, depending on the type of pain being treated. Ideally one drug is better than two drugs. Less site effect potential. For some types of pain, however, the use of two antagonists may be required.

The effect of PAF and the PAF antagonists is assessed on the release of prostaglandin E2 (PGE₂) from astrocytes. Also disclosed herein is the participation of the two COX isozymes in PAF-induced PGE₂ mobilization, using COX inhibitors with varying degrees of selectivity for COX-1 and COX-2. It has been suggested that activated astrocytes are responsible for the majority of the increased arachidonic acid and eicosanoid levels. The PAF-induced PGE₂ release initiates an inflammatory cascade in astrocytes that can be detrimental to cell function and/or kill surrounding neurons. By preventing such actions by endogenous PAF, PAF antagonists alleviate pain and provide neuroprotection in various disorders of the nervous system that are caused by or made worse by inflammatory mediator production.

PAF Antagonists

PAF antagonists include natural products (naturally occurring PAF-antagonists including chemical derivatives of terpenes, lignans and gliotoxins), synthetic structural analogs of PAF, synthetic PAF antagonist compounds that have thiazolidine/thiazole and pyridine moieties, synthetic PAF antagonist compounds that have methylimidazopyridine moieties, and synthetic small molecule PAF antagonists, and any other compounds that possesses the activity of PAF antagonist.

Natural Products

An example of naturally occurring PAF antagonists are the ginkgolides A, B, and C, T, and M. These compounds are terpenoids derived from the leaves of Ginkgo biloba, and are competitive PAF antagonists. The Ginkgo biloba tree of Gingkoaceae is of the gymnosperm order Ginkoales. Of these, ginkolide B is the strongest PAF antagonist, and is commercially available under the name BN52021 (IHB, Research Labs, France, among other commercial companies).

Plants of the Zingiberaceae species, including but not limited to, Alphinia galanga, Boesenbergia pandurata, Curcuma aeruginosa, C. domestica, C. ochorrhiza, C. xanthorriza, Aingiber officinale, and Z. zerumbet have effects similar to the Gingko Biloba extracts. Other sources of PAF antagonists include the cinnamomum species such as Cinnamomum altissimum, C. aureofulvum, and C. pubescens, as well as Ardisia elliptica, Goniothalamus malayanus, Kopsia flavida, Momordica charantia and Piper aduncem. Lastly, the bark extract of Drymis winteri of the Winteraceae family contains a sesquiterpene with anti-inflammatory and anti-allergic properties.

The best described ligand with PAF antagonist activity is kadsurenone (from Piper Futokadsurae, South China). It is orally active and is reported to have potent antagonist activity in a number of systems. A structural analogue, L-65 2731 (Merck Sharp and Dome) has considerably enhanced potency.

Fermentation of some fungi and micro-organism have produced antagonists which are structurally related to the gliotoxins. The most potent antagonist are FR 900 452 (S phacofaciens) and FR-49175 (F. testikowski).

PAF antagonists of the tetrahydrofuran class include L659,989 (trans-2-(3-methoxy-5-methylsulfonyl-4-propoxyphenyl)-5(3,4,5-trimethoxyphenyl) tetrahydrofuran); MK 287 (L-680,573); and magnone A ((2S,3R,4R)-tetrahydro-2-(3,4-dimethoxyphenyl)-4-(3,4-dimethoxybenzoyl)-3-(hydroxymethyl)furan) and magnone B ((2S,3R,4R)-tetrahydro-2-(3,4,5-trimethoxyphenyl)-4-(3,4-dimethoxybenzoyl)-3-(hydroxymethyl)furan) from the flower buds of Magnolia fargesii.

PAF antagonists of the benzodiazepine class include WEB-2086, WEB-2170, Y-24180, BN 50727, BN 50730, BN 50739, and E 6123. The triazolobenzodiazepines, particularly Alprazolam and Triazolam potently inhibit PAF activity in vitro. Structural alteration of the triazolobenzodiazepines has resulted in production of numbers of potent antagonists of which WEB 2086 (Boehringer Ingelhelm) is the most widely studied.

Synthetic Structural Analogs of PAF

Synthetic compounds with structures similar to PAF include CV-3988, CV-3938, CV-6209, TCV-309, E5880, and SRI 63-441. The most widely used and one of the first PAF antagonists developed is CV-3988 which incorporates an octadecyl carbamate in position 1, a methylether in position 2 and thiazolium ethyl phosphate in position 3. It is orally active in most systems tested and is relatively potent. At very high dose it may antagonise arachidonic acid and ADP activation of platelets.

Other Synthetic Structures

Synthetic compounds useful as PAF antagonists having thiazolidine/thiazole and pyridine moieties include SM-12502, YM264, ABT-299, SR 27417. Cyclization of the PAF structure has resulted in another series SRI 63-073 (Sandoz). A heptamethylene thiazolium at C.sub.3 gave a potent antagonist termed ONO-6240. Other minor alterations to this basic structure have been performed by Hoffman La Roche and RO-19 3704 is the best of these antagonists. Synthetic compounds useful as PAF antagonists having methylimidazopyridine moiety include UK-74,505 and BB-882 (Lexipafant). WEB 2086 (Apafant) is derived from an anxioilytic triazolobenzodiazepine. WEB 2086 related compounds include Y-24180, BN 50727, BN 50730, BN 50739, and E 6123. Lastly, GM2 activator protein is a good candidate for the development of small molecule PAF antagonists.

PAF-Mediated Conditions or PAF-Related Conditions

In another aspect, the present invention provides pharmaceutical compositions useful for the treatment of PAF-mediated disorders comprising a therapeutically effective amount of a PAF antagonist compound in combination with a pharmaceutically acceptable carrier.

In another aspect, the present invention provides a method of inhibiting PAF activity by administering to a host mammal in need of such treatment an effective amount of a PAF-antagonist compound.

In yet another aspect of the present invention, there is provided a method of treating PAF-mediated disorders or PAF-related disorder including ischemia and stroke, sepsis, inhibit the inappropriate or excessive contraction of the uterus, inhibit pain and/or cramps associated with premenstrual syndrome (also known as late luteal phase dysphoric disorder, or premenstrual dysphoric disorder), inhibit pain and/or cramps associated with normal menses, inhibit spontaneous abortion/miscarriage, inhibit pain, cramping, and/or discomfort associated with the perimenopausal period, reduce pain associated with childbirth, including pain experienced during and post labor, inhibit Braxton Hicks contractions, inhibit the initiation and/or the severity of septic shock, inhibit the proliferation of tumor cells, inhibit neo-angiogenesis by administering to a host mammal in need of such treatment a therapeutically effective amount of PAF antagonist compound.

Other important indications for a PAF antagonist include the following: epilepsy, extension of strokes after initial tissue damage, Alzheimer's disease, Parkinson's disease, Huntington's disease, functional brain damage secondary to primary and secondary brain tumors, Pick's disease, diffuse Lewy body disease, progressive supranuclear palsy, cerebellar degeneration, Shy-drager syndrome, amyotrophic lateral sclerosis, spinal muscular atrophy, multiple sclerosis, local brain damage secondary to meningitis or brain abscess, viral meningitis, viral encephalitis, HIV neurological disease, local brain damage secondary to trauma.

Yet other important indications for a PAF antagonist include the following: inflammatory processes of the tracheobronchial tree (acute and chronic bronchitis, bronchial asthma) or of the kidneys (glomerulonephritis), the joints (rheumatic complaints), anaphylactic conditions, allergies and inflammation in the mucous membranes (rhinitis, conjunctivitis) and the skin (e.g. psoriasis, atopic eczema, cold-induced urticaria, allergic dermatitis) and shock caused by sepsis, endotoxins, trauma or burns, lesions and inflammation in the gastric and intestinal linings, such as shock ulcers, ulcerative colitis, Crohn's disease, ischemic bowel necrosis, stress ulcers and peptic ulcers in general, but particularly ventricular and duodenal ulcers; obstructive lung diseases such as bronchial hyper-reactivity; inflammatory diseases of the pulmonary passages, such as chronic bronchitis; cardio/circulatory diseases such as polytrauma, anaphylaxis and arteriosclerosis; inflammatory intestinal diseases, EPH gestosis (edema-proteinuria hypertension); diseases of extracorporeal circulation, e.g. heart insufficiency, cardiac infarct, organ damage caused by high blood pressure, ischaemic diseases (i.e., cerebral, myocardial and renal ischemia), inflammatory and immunological diseases (i.e. rheumatoid arthritis); immune modulation in the transplanting of foreign tissues, e.g. the rejection of kidney, liver an other transplants; immune modulation in leukemia; propagation of metastasis, e.g. in bronchial neoplasia; diseases of the CNS, such as migraine, multiple sclerosis, endogenic depression and agarophobia (panic disorder). The PAF antagonist compounds of the present invention could also be effective as cyto- and organoprotective agents, e.g. for neuroprotection; to treat DIC (disseminated intravascular coagulation); to treat side effects of drug therapy, e.g. anaphylactoid circulatory reactions; to treat incidents caused by contrast media and other side effects in tumor therapy; to diminish incompatibilities in blood transfusions; to prevent fulminant liver failure (CCl₄ intoxication); to treat amanita phalloides intoxication (mushroom poisoning); to treat symptoms of parasitic diseases (e.g. worms); to treat autoimmune diseases (e.g. Werlhof s disease); to treat autoimmune hemolytic anemia, autoimmunologically induced glomerulonephritis, thyroids Hashimoto, primary myxoedema, pernicious anemia, autoimmune atrophic gastritis, Addison's disease, juvenile diabetes, Goodpasture syndrome, idiopathic leukopenia, primary biliary cirrhosis, active or chronically aggressive hepatitis (HBsAg-neg.), ulcerative colitis and systemic lupus erythematodes (SLE), idiopathic thrombocytopenic purpura (ITP); to treat diabetes, diabetic retinopathy, polytraumatic shock, haemorrhagic shock; to treat thrombocytopenia, endotoxin shock, adult respiratory distress syndrome; and to treat PAF-associated interaction with tissue hormones (autocoid hormones), lymphokines and other mediators; and any other condition in which PAF is implicated.

The PAF antagonist compounds of the present invention may also be used in combinations for which PAF-antagonists are suitable, e.g. with .beta.-adrenergics, parasympatholytics, corticosteroids, antiallergic agents and secretolytics. When the PAF antagonist compounds of the present invention are combined with TNF (tumor necrosis factor), the TNF is likely to be better tolerated (elimination of disturbing side effects). Thus, TNF may be used in higher dosages than when it is administered alone. The term “combination” here also includes the administration of the two active substances in separate preparations simultaneously or in sequence over a time period. When compounds are administered in combination with .beta.-adrenergics, a synergistic effect may be achieved.

Mode of Administration and Pharmaceutical Compositions

The compounds of the present invention include pharmaceutically acceptable salts that can be prepared by those of skill in the art. As used herein, by “pharmaceutically acceptable salt” it is meant those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M Berge, et al. describe pharmaceutically acceptable salts in detail in J. Pharmaceutical Sciences, 1977, 66: 1-19. The salts can be prepared in situ during the final isolation and purification of the compounds of the invention, or separately by reacting the free base function with a suitable organic acid. Representative acid addition salts include acetate, adipate, alginate, ascorbate, aspartate, benzene-sulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary as ammonium, and mine cations, including, but not limited to ammonium, tetramethylammonium, tetraethylammonium, methylamine, dimethylamine, trimethylamine, triethylamine, ethylamine, and the like.

The present invention also provides pharmaceutical compositions which comprise one or more of the PAF antagonist compounds described above formulated together with one or more non-toxic pharmaceutically acceptable carriers. The pharmaceutical compositions may be specially formulated for oral administration in solid or liquid form, for parenteral injection, or for rectal administration.

The pharmaceutical compositions of this invention can be administered to humans and other animals orally, rectally, parenterally, intracisternally, intravaginally, intraperitoneally, topically (as by powders, ointments, or drops), bucally, or as an oral or nasal spray. The term “parenteral” administration as used herein refers to modes of administration which include intravenous, intramuscular, intraperitoneal, intrathecally, intrasternal, subcutaneous and intraarticular injection and infusion.

Pharmaceutical compositions of this invention for parenteral injection comprise pharmaceutically acceptable sterile aqueous or nonaqueous solutions, dispersions, suspensions, or emulsions as well as sterile powders for reconstitution into sterile injectable solutions or dispersions just prior to use. Examples of suitable aqueous and nonaqueous carders, diluents, solvents, or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drag then depends upon its rate of dissolution which, in turn, may depend upon crystal size and crystalline form. Alternatively, delayed absorption of a parenterally administered drug form is accomplished by dissolving or suspending the drag in an oil vehicle.

Injectable depot forms are made by forming microencapsule matrices of the drag in biodegradable polymers such as polylactide-polyglycolide. Depending upon the ratio of drag to polymer and the nature of the particular polymer employed, the rate of drag release can be controlled. Examples of other biodegradable polymers include poly(orthoesters) and poly(anhydrides). Depot injectable formulations are also prepared by entrapping the drag in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions for rectal or vaginal administration are preferably suppositories which can be prepared by mixing the compounds of this invention with suitable nonirritating excipients or carriers such as cocoa butter, polyethylene glycol, or a suppository wax which are solid at room temperature but liquid at body temperature and therefore melt in the rectum or vaginal cavity and release the active compound.

The PAF antagonist compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a PAF antagonist compound of the present invention, stabilizers, preservatives, excipients, and the like. The preferred lipids are the phospholipids and the phosphatidyl cholines (lecithins), both natural and synthetic.

Methods to form liposomes are known in the art. See, for example, Prescott, Ed., Methods in Cell Biology, Volume XIV, Academic Press, New York, N.Y. (1976), p. 33 et seq.

Dosage forms for topical administration of a PAF antagonist compound of this invention include powders, sprays, ointments, and inhalants. The active compound is mixed under sterile conditions with a pharmaceutically acceptable carrier and any needed preservatives, buffers, or propellants which may be required. Opthalmic formulations, eye ointments, powders and solutions are also contemplated as being within the scope of this invention.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend as upon the activity of the particular PAF antagonist compound, the route of administration, the severity of the condition being treated, and the condition and prior medical history of the patient being treated. However, it is within the skill of the art to start doses of the PAF antagonist compound at levels lower than required to achieve the desired therapeutic effect and to gradually increase the dosage until the desired effect is achieved.

The pharmaceutical compositions of the present invention can be used in both veterinary medicine and human therapy. The magnitude of a prophylactic or therapeutic dose of the pharmaceutical composition of the invention in the acute or chronic management of pain associated with above-mentioned diseases or indications will vary with the severity of the condition to be treated and the route of administration. The dose, and perhaps the dose frequency, will also vary according to the age, body weight, and response of the individual patient. In general, the total daily dose range of the PAF antagonist compound of this invention is generally between about 0.001 to about 100 mg, preferably about 0.01 to about 20 mg, and more preferably about 0.1 to about 10 mg of active compound per kilogram of body weight per day are administered orally to a mammalian patient. If desired, the effective daily dose may be divided into multiple doses for purposes of administration, e.g. two to four separate doses per day.

Alternatively, the total daily dose range of the active ingredient of this invention is generally between about 1 and 500 mg per 70 kg of body weight per day, or about 10 and 500 mg per 70 kg of body weight per day, between about 50 and 250 mg per 70 kg of body weight per day, and more preferably between about 100 and 150 mg per 70 kg of body weight per day.

It is intended herein that by recitation of such specified ranges, the ranges cited also include all those dose range amounts between the recited range. For example, in the range about 1 and 500, it is intended to encompass 2 to 499, 3-498, etc, without actually reciting each specific range. The actual preferred amounts of the active ingredient will vary with each case, according to the species of mammal, the nature and severity of the particular affliction being treated, and the method of administration.

It is also understood that doses within those ranges, but not explicitly stated, such as 30 mg, 50 mg, 75 mg, etc. are encompassed by the stated ranges, as are amounts slightly outside the stated range limits.

Alternatively, the total daily dose range of the PAF antagonist compound of this invention is generally between about 10⁻⁸ and 10⁻³ molar range per 70 kg of body weight per day, or about 10⁻⁷ and 10⁻⁴ molar range per 70 kg of body weight per day, preferably between about 10⁻⁶ and 10⁻² molar range per 70 kg of body weight per day, and more preferably between about 10⁻⁵ and 10⁻¹ molar range per 70 kg of body weight per day. It is intended herein that by recitation of such specified ranges, the ranges cited also include all those concentration amounts between the recited range. For example, in the range about 10⁻⁸ and 10⁻³ molar range, it is intended to encompass 1.1×10⁻⁸ to 9.9×10⁻⁴, 1.2×10⁻⁸ to 9.8×10⁻⁴, etc, without actually reciting each specific range. The actual preferred amounts of the active ingredient will vary with each case, according to the species of mammal, the nature and severity of the particular affliction being treated, and the method of administration.

In general, the pharmaceutical compositions of the present invention are periodically administered to an individual patient as necessary to improve symptoms of the particular disease being treated. The length of time during which the compositions are administered and the total dosage will necessarily vary with each case, according to the nature and severity of the particular affliction being treated and the physical condition of the subject or patient receiving such treatment.

It is further recommended that children, patients aged over 65 years, and those with impaired renal or hepatic function initially receive low doses, and that they then be titrated based on individual response(s) or blood level(s). It may be necessary to use dosages outside these ranges in some cases, as will be apparent to those of ordinary skill in the art. Further, it is noted that the clinician or treating physician will know, with no more than routine experimentation, how and when to interrupt, adjust, or terminate therapy in conjunction with individual patient response.

The term “unit dose” is meant to describe a single dose, although a unit dose may be divided, if desired. Although any suitable route of administration may be employed for providing the patient with an effective dosage of the composition according to the methods of the present invention, oral administration is preferred. Suitable routes include, for example, oral, rectal, parenteral (e.g., in saline solution), intravenous, topical, transdermal, subcutaneous, intramuscular, by inhalation, and like forms of administration may be employed. Suitable dosage forms include tablets, troches, dispersions, suspensions, solutions, capsules, patches, suppositories, and the like, although oral dosage forms are preferred.

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

The present invention is illustrated by the Examples that follow, it being understood, however, that the invention is not limited to the specific details of these Examples.

### EXAMPLE ONE

Introduction

This study examines the effect of PAF and PAF analogs on the release of the pro-inflammatory mediator, prostaglandin E₂ (PGE₂), from rat cortical cell preparations enriched in astrocytes, an in vitro cell culture system believed to be a model for reactive astrocytes, (J. L. Ridet, et al., Reactive astrocytes: cellular and molecular cues to biologicalfunction, Trends Neurosci. 20 (1997) 570-577). PAF is readily hydrolyzed by extra- and intra-cellular PAF acetylhydrolases (PAF-AH), (for a review, see Z. S. Derewenda, et al., PAF-acetylhydrolases, Biochim. Biophys. Acta 1441 (1999) 229-236) therefore a non-hydrolyzable analog of PAF, methylcarbamyl-PAF (mc-PAF) is used for some experiments. The synthetic PAF analogs PAF-16 and PAF-18; the PAF precursor lyso-PAF; and the structurally similar lipids phosphatidylcholine (PC) and lyso-phosphatidylcholine (lyso-PC) are also assessed, to better determine the mechanism of PAF action. Whether co-incubation of AA and mc-PAF could have a synergistic effect on PGE₂ release is also assessed. Finally, the potential site(s) of PAF action is investigated, by examining the effect of specific PAF binding site antagonists on the mc-PAF-induced PGE₂ release.

Materials and Methods

Cell Culture

All experimental protocols are approved by the Massachusetts Institute of Technology institutional review committee and meet the guidelines of the National Institutes of Health. Dissociated astrocytes are cultured from cortices of postnatal day 1-2 rat pups (as described by K. D. McCarthy, et al., Preparation of separate astroglial and oligodendroglial cell cultures from rat cerebral tissue, J. Cell Biol. 85 (1980) 890-902, with minor modifications R. K. K. Lee, et al., Metabolic glutamate receptors increase amyloid precursor protein processing in astrocytes: inhibition by cyclic AMP, J. Neurochem. 68 (1997) 1830-1835.) In brief, cells from dissociated cortices are plated onto poly-L-lysine coated 35- or 100 mm culture dishes. The initial culture media, minimal essential medium (MEM, Gibco-Life Technologies; Rockville, Md.) containing 15% horse serum (BioWhittaker; Walkersville, Md.), are aspirated 2-5 h after plating to remove unattached cells and debris, and replaced with MEM containing 5% fetal bovine serum (FBS, BioWhittaker; Walkersville, Md.). Half the medium is replaced with MEM/5% FBS twice weekly. Astrocytes are kept at 37° C. in a humidified 5% CO₂/95% air incubator for 9-15 days, by which time the cultures are confluent and can be used for experiments.

Immunohistochemical procedures are carried out to more precisely identify the cell types in the cultures. Cells are fixed with 4% paraformaldehyde in 0.1 M phosphate buffered saline (PBS; pH 7.4) for 10 min, incubated in Chemiblock (Chemicon, Temecula, Calif.) solution for 1 h, and incubated with primary antibodies (CD-45, NF-145, NF-70 (1:1000) Calbiochem, La Jolla, Calif.), N-200 and GFAP (1:2,000 and 1:3000, respectively; Sigma, St. Louis, Mo.) overnight at room temperature on an orbital shaker. Cells are then incubated with a biotinylated secondary antibody for 30 min, followed by an incubation with ABC (Vector, Burlingtom, VR) solution for 30 min. Cells are then placed for 6 min in a 0.02% 3,3-diaminobenzadine tetrahydrochloride (DAB) solution containing H₂O₂ for visualization of the bound chromogen.

Most of the cells in this preparation (approximately 85% of cultured cells) are immunopositive for the astrocyte-specific intermediate filament protein glial fibrilary acidic protein (GFAP) and have the characteristics of flat type I-like astrocytes. It should be noted, however, that endothelial cells might also be immunopositive for GFAP, (F. A. Ghazanfari, et al., Characteristics of endothelial cells derived from the blood-brain barrier and of astrocytes in culture, Brain Res. 890 (2001) 49-65.)

The only other immunologically identifiable cells are microglia (approximately 5% of cultured cells are immunopositive for CD-45). No neurons are detected using neurofilament-specific antibodies. Many of the remaining cells exhibit a morphology reminiscent of radial glia that have not yet assumed the genetic program of mature astrocytes, (E. D. Laywell, et al., Identification of a multipotent astrocytic stem cell in the immature and adult mouse brain, Proc. Natl. Acad. Sci. 97 (2000) 13883-13888.)

Drug Preparation

Mc-PAF (Biomol; Plymouth Meeting, Pa.) is dissolved in methanol at a stock concentration of 10 mM. PAF-16, PAF-18, lyso-PAF, AA (Cayman Chemicals), lyso-PC and PC (Sigma) are dissolved in ethanol at stock concentrations of 10 mM. All stock solutions of lipids are stored at −80° C. and are used within 6 weeks of reconstitution. BN 52021 and CV 6209 (Biomol) are dissolved in ethanol. These PAF antagonists are stored at −20° C. in stock concentrations of 100 mM. BN 50730 (Biomeasure; Milford, Mass.) is dissolved in 45% hydroxy-B-cyclodextrin (HBC). All agents are diluted in warmed serum-free medium prior to cell stimulation. Equal amounts of vehicle are added to control cells.

Drug Treatments

Cells used for all experiments are established in vitro 9-15 days prior to use in experiments and are over 95% confluent. Serum-containing media are changed every 3-4 days. Cells are serum-deprived 24 hours prior to experimental treatments. Where treatment with PAF antagonists is indicated, these compounds are added 30 min prior to the addition of other agents.

PGE₂ Assay

PGE₂ levels are measured by ELISA according to manufacturer's instructions (Cayman Chemicals, Ann Arbor, Mich.). Since the amount of PGE₂ in fresh medium is negligible, (J. Luo, et al., Transforming growth factor B1 regulates the expression of cyclooxygenase in cultured cortical astrocytes and neurons, J. Neurochem. 71 (1998) 526-534), direct assays of the PGE₂ concentration in cell-conditioned medium is used as a measurement of PGE₂ secretion by cultured cells. Results are derived from at least 3 separate experiments, assayed in duplicate or triplicate (n=6-8). The reliable detection limit of this assay (i.e. sensitivity) varies across experiments and averages 14+/−6 pg of PGE₂.

Statistical Analysis

Data are expressed as means +/−SEMs. Statistical analyses are performed using unpaired Student's t-tests or ANOVAs for comparisons between groups, followed by Fischer's PLSD post hoc comparisons by means contrast. P values <0.05 are considered statistically significant.

Results

Effect of Solvents on Astrocytic PGE₂ Release

Addition of either methanol or ethanol (or a combination of both) to astrocyte-enriched cortical cell cultures causes an increase in PGE₂ release (less than a 10% increase) that is not statistically significant; HBC has no effect on PGE₂ release (data not shown).

mc-PAF Increases astrocytic PGE₂ Release in a Time-Dependent Manner

Addition of the non-hydrolyzable PAF analog mc-PAF (1 μM) to treatment media causes a time-dependent increase in PGE₂ release from astrocyte-enriched cortical cell cultures (FIG. 1). Within 5 min of mc-PAF incubation, an increase in PGE₂ release is observed (p<0.05). The maximum mobilization of PGE₂ occurs at 30 min (p<0.01), decreasing gradually by 4 hr. A second peak, albeit smaller, is observed at 8 hr (p<0.05), and levels returned to baseline by 12 hr. As the peak release of PGE₂ by mc-PAF is demonstrated to occur at 30 min, this time is used in subsequent studies to assess potential mechanisms of PAF-induced PGE₂ release.

PAF Analogs Increase Astrocytic PGE₂ Release in a Concentration-Dependent Manner

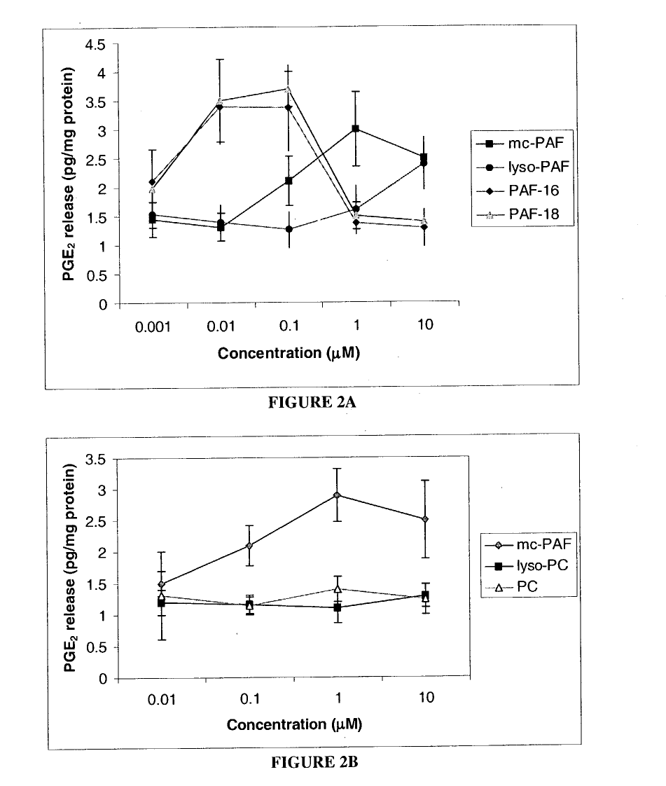

Addition of mc-PAF, lyso-PAF, PAF-16, or PAF-18 to astrocyte-enriched cortical cell cultures results in concentration-dependent increases in PGE₂ release into the conditioned media (FIG. 2A). Mc-PAF significantly increases PGE₂ release at concentrations of 0.1 (p<0.05), 1 (p<0.01), and 10 (p<0.01) μM, and lyso-PAF at a concentration of 10 (p<0.05) μM. Both PAF-16 and PAF-18 increase PGE₂ release at concentrations of 0.01 (p<0.05), and 0.1 (p<01) μM, but are less effective at higher concentrations (FIG. 2A).

Though treatment with PAF-16 or PAF-18, causes significant effects these effects are more variable across and within experiments than those produced by lyso-PAF or mc-PAF. For this reason, mc-PAF is used to explore the mechanisms mediating PAF-induced mobilization of PGE₂. Addition to the media of PC or lyso-PC, lipids, which are structurally similar to PAF analogs, has no effect on PGE₂ release at any concentration examined (10, 1, 0.1, and 0.01 μM; (FIG. 2B).

Arachidonic Acid and mc-PAF Act Synergistically to Increase Astrocytic PGE₂ Release

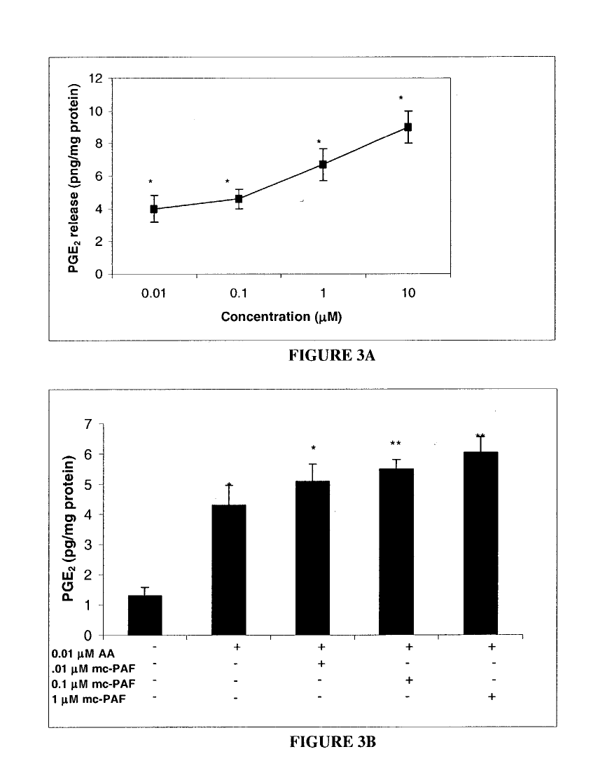

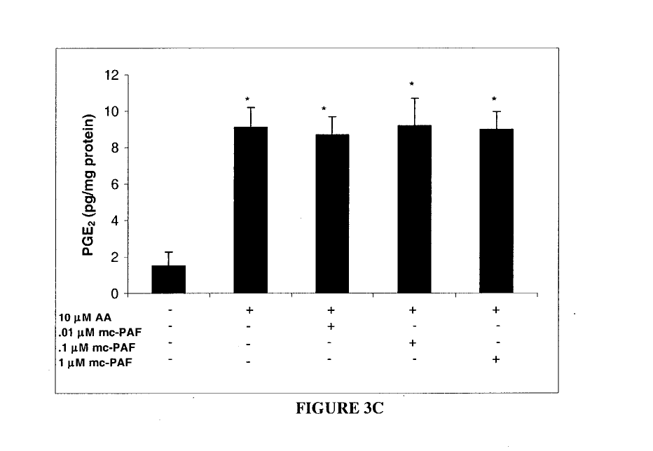

Treatment of astrocyte-enriched cell cultures for 30 min with AA (0.01-10 μM) increases PGE₂ release (p<0.01; FIG. 3A). Co-administration of AA with mc-PAF (0.1, 1 or 10 μM) causes an additive increase in PGE₂ release with a low arachidonate concentration (0.01 μM) (p<0.05; FIG. 3B), but not at a high AA concentration (10 μM) (FIG. 3C). These results suggest a “ceiling effect” might block added responses to higher AA concentrations (i.e. no synergism), perhaps mediated by limits in the availability of downstream enzymes responsible for AA conversion to PGE₂ (e.g. cyclooxygenases).

Effect of Intracellular PAF Binding Site Antagonists on PAF Analog- and AA-Induced PGE₂ Release

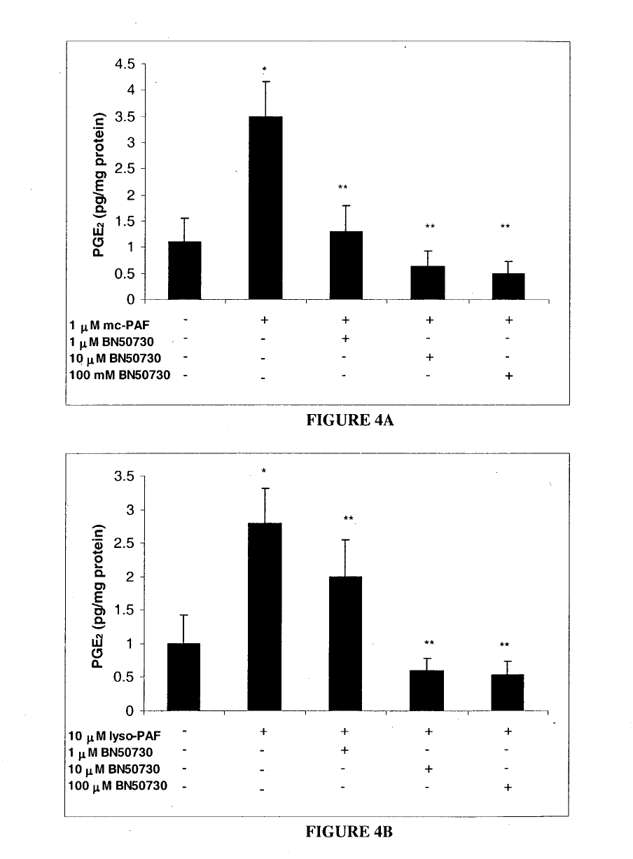

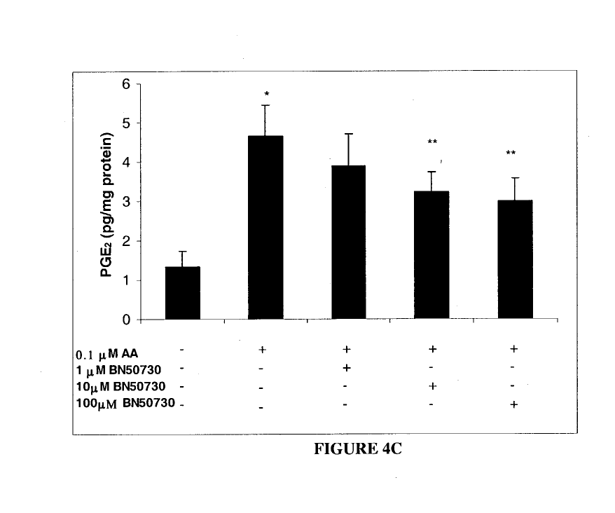

Prior exposure of cells to BN 50730(0.1-100 μM) attenuates mc-PAF-induced PGE₂ release (FIG. 4A). Prior administration of BN 50730 also significantly attenuates the increase in PGE₂ release induced by lyso-PAF (FIG. 4B). These results suggest that intracellular PAF binding sites are essential for the PAF analog-induced effect on PGE₂ mobilization. Prior exposure of cells to BN 50730 also significantly attenuates the release of PGE₂ induced by AA (FIG. 4C), suggesting that exogenous AA might increase intracellular PAF.

Effect of Cell Surface PAF Antagonists on PAF Analog- and AA-Induced PGE₂ Release

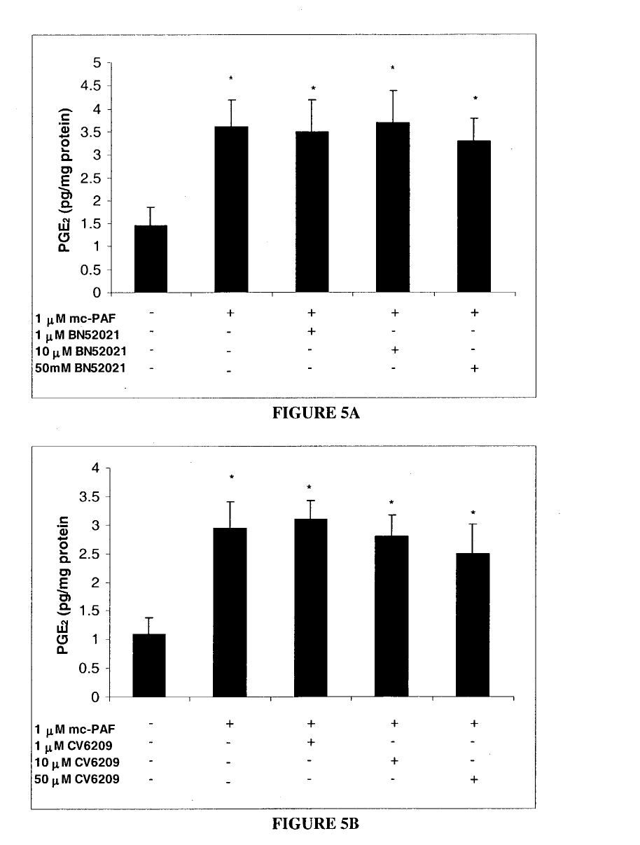

BN 52021 and CV 6209, two structurally distinct antagonists to cell surface PAF receptors, have no significant effect on mc-PAF-induced PGE₂ release (FIGS. 5A, 5B, respectively) at concentrations previously shown to effectively block the plasma membrane receptors, (V. L. Marcheselli, et al., Distinct platelet-activating factor binding sites in synaptic endings and in intracellular membranes of rat cerebral cortex, J. Biol. Chem. 265 (1990) 9140-9145.) At higher concentrations both antagonists attenuate by 20-25% the mc-PAF-induced increase in PGE₂; this effect could be caused by blockade at intracellular sites. These agents have no effect on the PGE₂ release caused by lyso-PAF-or AA (data not shown). When either BN 52021 or CV 6209 is administered alone (i.e. no mc-PAF), PGE₂ release is increased, perhaps by shunting endogenous PAF to intracellular binding sites.

Discussion

These data show that PAF enhances PGE₂ release from cortical astrocytes; that mc-PAF and lyso-PAF share this effect; that related phosphatides (PC, lyso-PC) fail to enhance PGE₂ release; that AA synergizes the effect of mc-PAF on PGE₂ production; and that intracellular PAF antagonists can attenuate the PGE₂ response elicited by PAF analogs and AA.

Increasing the concentration of mc-PAF (0.001-10 μM) causes an increase in the amounts of PGE₂ released into the media (FIG. 2A). The highest concentration of mc-PAF used in this study (10 μM) increases PGE₂ release, however greater variability was observed with some cultures displaying no increases in PGE₂ release. Incubation of cells with this high concentration for 24 hours causes cytologic evidence of toxicity. As 1 μM mc-PAF does not appear to have toxic effects and produces a reliable PGE₂ increase that varies very little across cultures (relative to other concentrations), this concentration is used to explore the site of PAF action. In contrast to mc-PAF's concentration-response effect on PGE₂ release, peak PGE₂ release is observed with 0.1 μM PAF-16 or PAF-18, and higher and lower concentrations of these compounds elicit less release (FIG. 2A). While it is unlikely that cell death occurs within 30 min, it is possible that these higher concentrations elicit a cellular program distinct from the physiological program activated by lower concentrations. Also, higher concentrations of synthetic PAF might have resulted in poor solubility or extracellular micelle formation causing less PAF to enter the cells.

PC and lyso-PC, which have similar abilities to perturb membranes, fail to affect PGE₂ release (FIG. 2B), suggesting that the PAF, mc-PAF and lyso-PAF effects are a result of specific actions on PAF binding sites, rather than nonspecific membrane perturbation. As lyso-PAF does not activate cell surface PAF receptors, (N. G. Bazan, et al., Bioactive lipids in excitatory neurotransmission and neuronal plasticity, Neurochem. Inter. 30 (1997) 225-231), this lipid may cause PGE₂ release by its conversion to intracellular PAF. Several lines of evidence support this hypothesis. First, lyso-PAF levels may be limiting in the remodeling pathway for PAF biosynthesis (E. Francescangeli, et al., Properties and regulation of microsomal PAF-synthesizing enzymes in rat brain cortex, Neurochem. Res. 25 (2000) 705-713.) Second, PAF and related analogs increase intracellular Ca²⁺ levels in astrocytes, (E. Fuentes, et al., Lysophospholipids trigger calcium signals but not DNA synthesis in cortical astrocytes, Glia 28 (1999) 272-276), and lyso-PAF-AH is fully active at the μM Ca²⁺ concentrations, (E. Francescangeli, et al., Properties and regulation of microsomal PAF-synthesizing enzymes in rat brain cortex, Neurochem. Res. 25 (2000) 705-713), induced by these compounds. Finally, lyso-PAF is able to enter cells by diffusion through plasma membranes, (E. Botitsi, et al., Metabolic fate of platelet-activating factor (PAF, 1-O-alkyl-2-acetyl-sn-3-phosphocholine) and lyso-PAF (1-O-alkyl-2-lyso-sn-glycerol-3-phosphocholine) in FRTL5 cells, J. Lipid Res. 39 (1998) 1295-1304). In fact, as the G-protein coupled PAF receptors do not appear to be critical for the effect of the PAF-analogs on PGE₂ release, these effects might be due in part, to hydrolysis to lyso-PAF, which might prevent the lipids from reaching intracellular sites.

BN 50730, a competitive antagonist to intracellular PAF binding sites, prevents mc-PAF-induced PGE₂ release (FIG. 4A). This finding is consistent with a previous study that suggests a role for intracellular PAF in the promotion of PGE₂ synthesis, (S. I. Svetlov, et al., Regulation of platelet-activating factor (PAF) biosynthesis via coenzyme A-independent transacylase in the macrophage cell line IC-21 stimulated with lipopolysaccharide, Biochim. Biophys. Acta. 1346 (1997) 120130). Lipopolysaccharide (LPS) rapidly increases both PGE₂ release, (R. Asmis, et al., PAF stimulates cAMP formation in P388D1 macrophage-like cells via the formation and secretion of prostaglandin E2 in an autocrine fashion, Biochimica et Biophysica Acta. 1224 (1994) 295-301), as well as intracellular PAF levels in macrophage cell lines (S. I. Svetlov, et al., Regulation of platelet-activating factor (PAF) biosynthesis via coenzyme A-independent transacylase in the macrophage cell line IC-21 stimulated with lipopolysaccharide, Biochim. Biophys. Acta. 1346 (1997) 120-130). Accumulation of PAF is accompanied by initial activation of cPLA₂ (within 5 min), followed by lyso-PAF-AT activation. These findings not only support a role for PAF in PGE₂ release, but also suggest that the enzyme responsible for lyso-PAF conversion to PAF is also activated early in LPS-induced PGE₂ release.

BN 50730 also attenuates PGE₂ release induced by lyso-PAF (FIG. 4B) and AA (FIG. 4C). While BN 50730 completely abolishes lyso-PAF and mc-PAF generated PGE₂ release, it only attenuates the AA-induced PGE₂ release. This suggests that intracellular PAF binding sites are required for the effects of mc-PAF and lyso-PAF on PGE₂ release, but not for those of AA. As AA can induce PGE₂ release even when these intracellular PAF binding sites are blocked, this suggests that the sites are not necessary for PGE₂ production when exogenous AA is made available to cells. However, the blockade of intracellular PAF binding sites does attenuate some of the mobilization of PGE₂ by AA. This attenuation may be explained by other actions of exogenous AA on cells. For instance, AA has previously been shown to increase cPLA₂ activation, (V. Di Marzo, Arachidonic acid eicosanoids as targets and effectors in second messenger interactiona, Prostagldns., Leukot., Essen. Fatty Acids. 53 (1995) 239-254). Thus, besides providing the necessary substrate for PGE₂ synthesis, exogenous AA can also produce more AA (and PAF) by activating cPLA₂. It is of interest to note, that in the case of lyso-PAF and mc-PAF, higher concentrations of BN 50730 not only attenuate the PAF analog-induced PGE₂ release but also reduce the basal release of PGE₂, suggesting that endogenous intracellular PAF has a role in basal PGE₂ release.

CV-6209 and BN 52021, which are structurally distinct antagonists to PAF's plasma membrane receptors, do not significantly influence mc-PAF-induced PGE₂ release (FIGS. 5A and 5B). Administration of these agents alone increases PGE₂ release (data not shown). Not to be limited by theory, this effect may be caused by a compensatory increase in PAF synthesis and/or a shunting of endogenously produced PAF to intracellular sites.

Our observation that PAF increases the released PGE₂ is in accordance with the results of previous studies. For instance, PAF increases the release of PGE₂ in trout astrocytes (2 h incubation), (D. R. Tocher et al., Production of eicosanoids derived from 20;4n-6 and 20:5n-3 in primary cultures of turbot (Scopthalmus maximus) brain astrocytes in response to platelet-activating factor, substance P and interleukin-1B, Comp. Biochem. Physiol. 115B (1996) 215-222), and PAF increases the release of other eicosanoids in mammalian astrocytes (15 min incubation), (A. M. Petroni, et al., Arachidonic acid cyclo and lipoxygenase pathways in astroglial cells. In: B. Samuelsson, et al. (Eds.), Advances in Prostaglandin, Thromboxane, and Leukotriene Research, Vol, 21B, Raven Press, New York, 1990, pp 743-747). Marked increases in AA levels and eicosanoids (including PGE₂) have been observed in association with brain inflammation, (S. Oka, et al., Inflammatory factors stimulate expression of group II

phospholipase A₂ in rat cultured astrocytes, J. Biol. Chem. 266 (1991) 9956-9960), and in degenerative disorders like HIV dementia, (D. E. Griffin, et al., Elevated central nervous system prostaglandins in human immunodeficiency virus-associated dementia, Ann. Neurol. 35 (1994) 592-597), ALS, and AD. It is thus proposed herein, that the PAF-induced PGE₂ release initiates an inflammatory cascade in astrocytes that can be detrimental to central nervous system function.

Summary

The phospholipid mediator platelet-activating factor (PAF) increases the release of prostaglandin E₂ (PGE₂) from astrocyte-enriched cortical cell cultures in a concentration- and time-dependent manner. The non-hydrolyzable PAF analog methylcarbamyl-PAF (mc-PAF), the PAF intermediate lyso-PAF, and arachidonic acid (AA) also produce this effect. In contrast, phosphatidlycholine (PC) and lyso-PC, lipids that are structurally similar to PAF and lyso-PAF, have no effect on PGE₂ production, suggesting that PAF-induced PGE₂ release is not the consequence of nonspecific phospholipid-induced membrane perturbation. Antagonism of intracellular PAF binding sites completely abolishes the ability of mc-PAF and lyso-PAF to mobilize PGE₂, and attenuates the AA effect. Antagonism of the G-protein-coupled PAF receptor in plasma membranes has no significant effect on mc-PAF, lyso-PAF or AA-induced PGE₂ release. It is thus proposed that intracellular PAF is a physiologic stimulus of PGE₂ production in astrocytes.

### EXAMPLE TWO

Introduction

The formalin test, a commonly used model of inflammatory nociception in rats, which elicits a biphasic behavioral response, (Dubuisson D, et al., The formalin test: a quantitative study of the analgesic effects of morphine, meperidine, and brain stimulation of rats and cats. Pain 4 (1977)161-174), is used to assess the involvement of PAF in nociception. The early phase starts immediately after injection of formalin, lasts about 5 min, and is thought to result from direct chemical stimulation of nociceptive fibers, (Jongsma et al., Markedly reduced chronic nociceptive response in mice lacking the PAC1 receptor. NeuroReport 12 (2001) 2215-2219). The late phase is exhibited 15-70 min after formalin injection and appears to depend on the combination of an inflammatory reaction in the peripheral tissue and functional changes in the dorsal horn of the spinal cord, (Tjolsen et al., The formalin test: an evaluation of the method. Pain 51 (1992) 5-17). To investigate the role of PAF in nociception, and the potential site(s) of its action, two structurally distinct PAF antagonists are administered systemically to rats 40 min prior to formalin injection, and their effects on the biphasic formalin response are measured. BN 52021 is a competitive PAF antagonist that selectively inhibits the cell surface PAF receptor, while BN 50730 is a specific inhibitor for intracellular PAF binding sites (Marcheselli et al., Distinct platelet-activating factor binding sites in synaptic endings and in intracellular membranes of rat cerebral cortex. J Biol Chem 265 (1990) 9140-9145; Marcheselli et al., Platelet-activating factor is a messenger in the electroconvulsive shock-induced transcriptional activation of c-fos and zif-268 in hippocampus. J Neurosci Research 37 (1994) 54-61.

Materials and Methods

Animals

Sixty male Sprague Dawley rats weighing 300-350 g (Taconic Labs, Canada) are housed in groups of 2-3 per cage, in polycarbonate cages. Animals are maintained under standard environmental conditions (room temperature: 20-20° C.; relative humidity: 55-60%; light/dark schedule: 12/12 hr) with free access to standard laboratory chow and tap water.

Drug Preparation and Administration

BN 50730 (Biomeasure; Milford, Mass.) and BN 52021 (Biomol) are dissolved in 45% hydroxypropyl-B-cyclodextrin in distilled water (HBC). Drugs (at doses of 10, 1, or 0.1 mg/kg) or vehicle are administered intraperitoneally (i.p.) 40 minutes prior to formalin injection. The doses chosen are based on those found, in previous studies, to produce central effects after their peripheral administration, (Bito et al., Characterization of platelet-activating factor (PAF) receptor in the rat brain. J Lipid Med 6 1993) 169-174; Marcheselli et al., Platelet-activating factor is a messenger in the electroconvulsive shock-induced transcriptional activation of c-fos and zif-268 in hippocampus. J Neurosci Research 37 (1994) 54-61).

Formalin Test

Experiments are carried out in accordance with The National Institutes of Health Guide for the Care and Use of Laboratory Animals. Behavioral testing is carried out in a blind manner. Nociceptive responses are examined in the formalin test described previously, (Dubisson et al., The formalin test: a quantitative study of the analgesic effects of morphine, meperidine, and brain stimulation of rats and cats. Pain 4 (1977) 161-174). In brief, animals are placed in a clear Plexiglas® formalin test box (30 cm×30 cm×30 cm), with a mirror positioned at a 45° angle below the floor allowing for unobstructed observation of the animal's paw. Following a 10 minute habituation period, animals are removed from the formalin box, at which time 50 μl of 1% formalin was injected subcutaneously (s.c.) into the plantar surface of the right hind paw with a 27-gauge needle. The amount of time that each rat elevated the injected paw is recorded in five-minute intervals during the 70-minute period following formalin injection. Each animal is used once.

The 60-minute formalin test produces a biphasic response consisting of an initial, rapidly decaying acute phase (early phase, 1-10 min after injection) followed by a slow rising and long-lived tonic phase (late phase, 15-60 min after injection). Typically animals elevate their paws following injection (i.e. the early phase) followed by a reduction in this behavior. Approximately 15-20 minutes after injection, the inflammatory late phase begins and animals again elevate their paws to varying degrees for the remainder of the testing period. The amount of time animals elevate their injected paw is used as a behavioral measure of pain.

Data Analysis

Data are expressed as means +/−SEM and p values <0.05 are considered statistically significant. Treatment groups are compared with vehicle-controls using one-way analysis of variance (ANOVA) followed by Fischer's PLSD post-hoc test to compare between groups if overall significance is found by ANOVA.

Results

BN 52021 Effects on Formalin-Induced Nociception

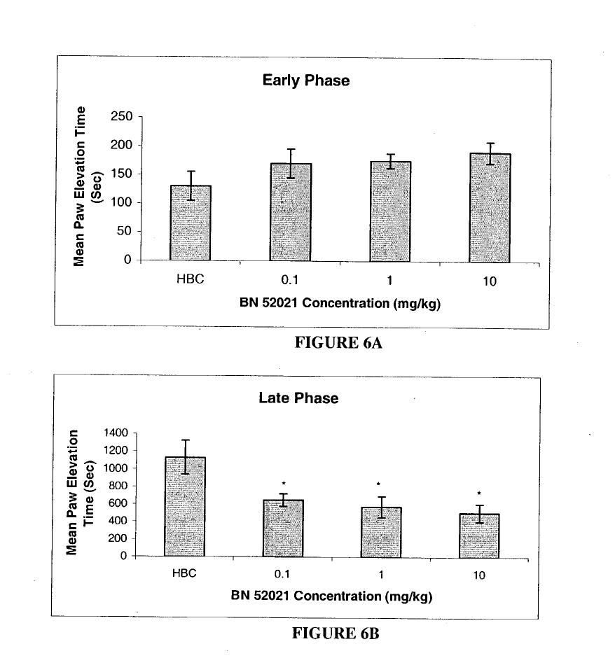

The nociceptive response (measured as time spent with the injected paw elevated) during the early phase (1-10 min post-formalin) is not significantly affected by BN 52021 administration (FIG. 6A) although rats that receive BN 52021 tend to elevate their paws for longer periods of time than do vehicle-treated controls. During the late phase (10-60 min), BN 52021-treated rats elevate their paws for significantly shorter times than do control-treated rats [F(3,30)=3.831, p<0.05; FIG. 1B]. Fisher's PLSD post-hoc analysis reveal that the responses of rats receiving 10 (p=0.008), 1 (p=0.013) or 0.1 (p=0.0366) mg/kg BN 52021 differs significantly from those of control-treated rats.

BN 50730 Effects on Formalin-Induced Nociception

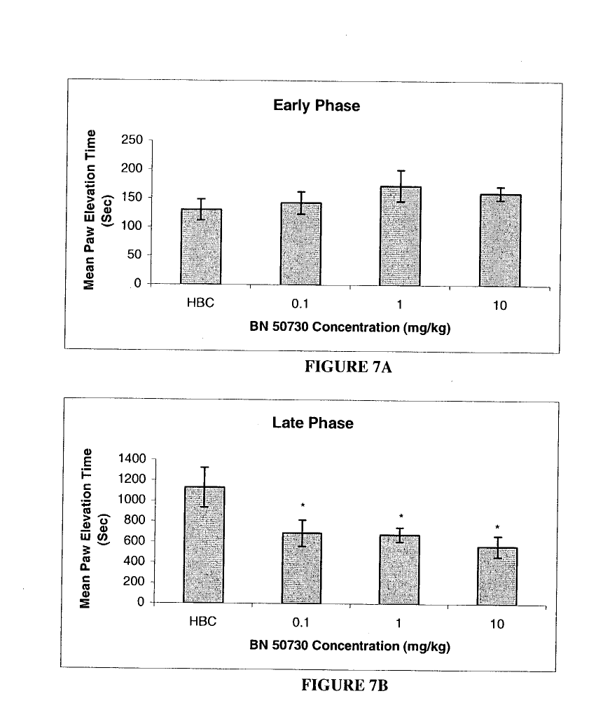

The nociceptive response (measured as time spent with the injected paw elevated) during the early phase (1-10 min post-formalin) is not significantly affected by BN 50730 administration (FIG. 7A). During the late phase (10-60 min), BN 50730-treated rats exhibit significantly shorter paw elevation times than do control-treated rats [F(3,30)=2.933, p<0.05; FIG. 6B]. Fisher's PLSD post-hoc analysis reveal that the behavior of rats receiving 10 (p=0.016), 1 (p=0.046) or 0.1 (p=0.049) mg/kg BN 50730 differ significantly from those of control-treated rats.

Discussion

These data show that systemic administration of PAF antagonists, which act selectively on cell surface or intracellular PAF binding sites (BN 52021 and BN 50730, respectively), decrease nociceptive behavior during the late, but not the early, phase of the formalin test in rats (FIGS. 6B and 7B). Although three doses are used for each antagonist a dose-response relationship is not revealed for either drug (i.e. all three doses of BN 52021 and BN 50730 decrease nociceptive behavior in a similar fashion). These results attest to the significance of endogenous PAF in nociception. PAF is extremely potent and tightly regulated; the lowest dose of each antagonist is likely sufficient to block enough of the the binding sites to prevent endogenous PAF from carrying out it's nociceptive function(s) at both intracellular and plasma membrane sites.

A feature of the formalin test in rodents is that animals show two distinct phases of nociceptive behavior, which seem to depend on different mechanisms, (Dubisson et al., The formalin test: a quantitative study of the analgesic effects of morphine, meperidine, and brain stimulation of rats and cats. Pain 4 (1977) 161-174). Substance P and bradykinin participate in the early phase, while histamine, serotonin and prostanoids appear to be involved in the late phase, (Shibata et al., Modified formalin test: characteristic pain response. Pain 29 (1989) 375-386). The early phase of formalin-induced nociception (also known as the acute phase) starts immediately after its injection, and is thought to result from direct chemical stimulation of chemosensitive nociceptors, (Dubisson et al., The formalin test: a quantitative study of the analgesic effects of morphine, meperidine, and brain stimulation of rats and cats. Pain 4 (1977) 161-174; Hatakeyama et al., Differential nociceptive responses in mice lacking the α1B subunit of N-type Ca²⁺ channels. NeuroReport 12 (2001) 2423-2427; Jongsma et al., Markedly reduced chronic nociceptive response in mice lacking the PAC1 receptor. NeuroReport 12 (2001) 2215-2219). The second phase (also known as the tonic phase) is thought to result from peripheral inflammatory processes, and from sensitization in the spinal cord produced by the first phase, (Tjolsen et al., The formalin test: an evaluation of the method. Pain 51 (1992) 5-17), as well as from functional changes in central processing, (Coderre et al., Central nervous system plasticity in the tonic pain response to subcutaneous formalin injection. Brain Res 535 (1990) 155-158). As both antagonists tend to increase nociceptive responses (albeit not significantly, FIGS. 6A and 7A) during the early phase, the decrease in nociceptive responses during the late phase cannot be attributed to a reduction in the early phase of formalin-induced nociception.

The use of peripheral injections as the means of administering the PAF antagonists does not allow conclusions to be drawn concerning their sites of action in attenuating late phase nociceptive responses. Not to be limited by theory, cell surface PAF receptor mRNA expression, (Mori et al., Predominant expression of platelet-activating factor receptor in the rat brain microglia. J Neurosci 16 (1996) 3590-3600) and the density of PAF binding sites (Bito et al., Characterization of platelet-activating factor (PAF) receptor in the rat brain. J Lipid Med 6 (1993) 169-174) are predominant in the cerebral cortex and hippocampus, and there is considerable evidence suggesting the involvement of the hippocampus in pain processing in humans, (Ploghaus et al., Learning about pain: The neural substrate of the prediction error for aversive events. Proc Natl Acad Sci 97 (2000) 9281-9286; Wei et al., Role of EGR1 in hippocampal synaptic enhancement induced by tetanic stimulation and amputation. J Cell Biol 149 (2000) 1325-1333), and nociceptive behaviors in rodents, (Blanchard et al., Effects of limbic lesions on passive avoidance and reactivity to shock. J Comp Physiol Psychol 66 (1968) 606-612; Prado et al., An assessment of the antinociceptive and aversive effects of stimulating identified sites in the rat brain. Brain Res 340 (1985) 219-228; Yeung, et al., Concurrent mapping of brain sites for sensitivity to the direct application of morphine and focal electrical stimulation in the production of antinociception in the rat. Pain 4 (1977) 23-40). Moreover, the hippocampus is known to have a mediatory role in the late, but not the early, phase of formalin-induced nociception, (McKenna et al, Analgesia produced by lidocaine microinjection into the dentate gyrus. Pain 49 (1992) 105-112). Thus, the antinociceptive effect of PAF antagonists during the late phase may have resulted from blockade of hippocampal PAF binding sites. PAF also exerts a variety of biological effects through actions at intracellular and cell surface binding sites in hippocampus, (for a review, see Bazan, Platelet-activating factor is a synapse messenger and an intracellular modulator of gene expression. J Lipid Med Cell Signal 10 (1994) 83-86; Bazan et al., Platelet-activating factor and intracellular signaling pathways that modulate gene expression. In: Platelet-Activating Factor Receptors: Signal Mechanisms and Molecular Biology (ed. S. Shukla), (1993) pp. (137-146). CRC Press Inc., Boca Raton, and Bazan et al., Bioactive lipids in excitatory neurotransmission and neuronal plasticity. Neurochem Intl 30 (1997) 225-231).

The antinociceptive effects of BN 52021 and BN 50730 may also result from blockade of the actions of endogenous PAF within the spinal cord, or at peripheral nervous system sites. Peripheral inflammation activates dorsal horn astrocytes (i.e. upregulated expression of activation markers), (Fu et al., Relationship between nociceptor activity, peripheral edema, spinal microglial activation and long-term hyperalgesia induced by formalin. Neuroscience 101 (2000) 1127-1135) and activated astrocytes maintain late, but not early, phase pain, (for review, see Watkins et al., Glial activation: a driving force for pathological pain. Trends Neurosci 24 (2001) 450-455) possibly by releasing prostaglandins (and other proinflammatory mediators), (Watkins et al., Evidence for the involvement of spinal cord glia in subcutaneous formalin induced hyperalgesia in the rat. Pain 71 (1997) 225-235). As disclosed herein, PAF increases prostaglandin E2 (PGE2) release from astrocytes, suggesting that PAF antagonists decrease nociceptive responses in the late phase by decreasing astrocytic PGE2 release in the dorsal horn of the spinal cord.

In conclusion, the nociceptive responses to subcutaneous formalin injection are significantly reduced in rats receiving PAF antagonists that act on intracellular or cell surface PAF binding sites. Selective PAF antagonists might thus be effective in the treatment of certain forms of acute and chronic pain.

Summary

Platelet-activating factor (PAF) is a membrane-derived phospholipid mediator that has biological effects on a variety of cells and tissues. A variety of stimuli, including those producing inflammation, promote the synthesis and release of PAF from various cell types. Evidence suggests that PAF exerts cellular actions through a plasma membrane receptor as well as via intracellular (microsomal) PAF binding sites. This second example: 1) investigates the role of PAF in a model of inflammatory nociception in rats (i.e. the formalin test), and 2) localizes PAF's site(s) of action in nociception. The effect of administering two PAF antagonists (BN 52021 and BN 50730, which are selective for cell surface and intracellular PAF binding sites, respectively) is assessed on formalin-induced nociceptive responses. As described in materials and methods above, forty minutes prior to formalin injection into the rat hindpaw, male Sprague Dawley rats receive systemic injections of BN 52021 (10, 1, or 0.1 mg/kg), BN 50730 (10, 1, or 0.1 mg/kg), or vehicle (45% 2-hydroxypropyl-B-cyclodextrin in distilled water, HBC) and the effects of the drugs on nociceptive behavioral responses is measured. Rats receiving systemic BN 52021 or BN 50730 display a significant reduction of nociceptive responses in the late, but not early, phase of formalin-induced nociception. These findings suggest a role for endogenous PAF in nociceptive transmission, especially for persistent pain like that which occurs in the late phase of the formalin test. The findings also indicate that both intracellular and cell surface PAF binding sites are involved in nociceptive modulation in rats, and that PAF antagonists are useful for treating some patients with acute or chronic pain.

### EXAMPLE THREE

Introduction

Prostaglandins (PGs) have important functions in brain cells, and may mediate a variety of neuropathologic phenomena, including such inflammation-associated disorders as Alzheimer's disease (AD) [Breitner, J. C. S., Gau, B. A., Welsh, K. A., Plassman, B. L., McDonald, W. M., Helms, J. J. and C, A. J., Inverse association of anti-inflammatory treatments and Alzheimer's disease: Initial results of a co-twin control study, Neurology, 44 (1994) 227-232] and amyotrophic lateral sclerosis (ALS) [Almer, G. A., Guegan, C., Teismann, P., Naini, A., Rosoklija, G., Hays, A. P., Chen, C. and Przedborski, S., Increased expression of the pro-inflammatory enzyme cyclooxygenase-2 in amyotrophic lateral sclerosis, Ann. Neurol., 27 (2001) 397-406]. When cells and tissue are exposed to various pro-inflammatory stimuli, arachidonic acid (AA) is liberated from membrane phospholipids and is converted to PGs, by the action of cyclooxygenase (COX) enzymes. Two related but unique isoforms of COX, cyclooxygenase-1 (COX-1) and cyclooxygenase-2 (COX-2) have been identified; both catalyze identical reactions, a cyclooxygenation to form PGG₂, and a peroxidation which reduces PGG₂ to PGH₂, the precursor of all other PGs, including PGE₂. COX-1 is constitutively expressed by most cells and is generally considered to be involved in maintaining cell homeostasis; in contrast the mitogen-inducible COX-2 is implicated in inflammatory and immune responses [Vane, J. R., Bakhl, Y. S. and Botting, R. M., Cyclooxygenases 1 and 2, Ann. Rev. Pharmacol. Toxicol., 38 (1998) 97-120].

A number of observations suggest that astrocytes have an important role in CNS inflammation/immune responses. Following CNS injury or an immune/inflammatory challenge, astrocytes undergo a phenotypic alteration—a response known as activation. The activated astrocytes then release cytokines and other pro-inflammatory mediators, including PGs. These released substances communicate with (and ultimately affect the function of) such neighboring cells as neurons and microvascular cells. Astrocytes are a major source of PGs in the CNS; in culture these cells synthesize up to 20 times more PGs than do neurons [Seregi, A., Keller, M., Jackisch, R. and Hertting, G., Comparison of the prostanoid synthesizing capacity in homogenates from primary neuronal and astroglial cell cultures, Biochem. Pharmacol., 33 (1984) 3315-3318]. PGE₂ is the major AA metabolite involved in modulation of immuno-inflammatory responses [Vane, J. R., Bakhl, Y. S. and Botting, R. M., Cyclooxygenases 1 and 2, Ann. Rev. Pharmacol. Toxicol., 38 (1998) 97-120].

The acute or immediate phase of inflammation is the earliest response to tissue injury, as well as to immunological or pro-inflammatory challenges. It has been shown in several cell types that COX-1 is often responsible for the immediate increases in PGs produced by various types of inflammatory stimuli, and COX-2 for the increased levels characteristic of the delayed phase of inflammation [Kuwata, H., Nakatani, Y., Murakami, M. and Kudo, I., Cytosolic phospholipase A₂ is required for cytokine-induced expression of type IIA secretory phospholipase A₂ that mediates optimal cyclooxygenase-2-dependent delayed prostaglandin E₂ generation in rat 3Y1 fibroblasts, J. Biol. Chem., 273 (1998) 1733-1740]. However other data suggest that the degree to which each COX isoform contributes to particular acute inflammatory responses depends upon such factors as the nature of the inflammatory stimulus and the cell type involved. We have recently shown that the pro-inflammatory mediator PAF increases the release of PGE₂ from cortical astrocytes [Teather, L. A., Lee, R. K. K. and Wurtman, R. J., Platelet-activating factor increases prostaglandin E2 release from astrocyte-enriched cortical cell cultures, Brain Res., 946 (2002) 87-95]. This effect is observed within minutes of PAF stimulation and PGE₂ accumulation peaks at 30 minutes, suggesting that PAF induces an acute inflammatory reaction in astrocytes. In the present study we examine the participation of two COX isozymes in PAF-induced PGE₂ mobilization, using COX inhibitors with varying degrees of selectivity for COX-1 and COX-2. Example Three examines the involvement of the COX isoforms in PAF-induced PGE₂ release.

Materials and Methods

Cell Culture

Dissociated astrocytes were cultured from cortices of postnatal day 1-2 rat pups as previously described [McCarthy, K. D., de Vellis, J., Preparation of separate astroglial and oligodendroglial cell cultures from rat cerebral tissue, J. Cell Biol., 85 (1980) 890-902] with minor modifications [Teather, L. A., Lee, R. K. K. and Wurtman, R. J., Platelet-activating factor increases prostaglandin E2 release from astrocyte-enriched cortical cell cultures, Brain Res., 946 (2002) 87-95]. In brief, cells from dissociated cortices were plated onto poly-L-lysine coated 35- or 100 mm culture dishes. All cell culture constituents were purchased from Gibco-Life Technologies (Rockville, Mass.). The initial culture media, minimal essential medium (MEM) containing 15% horse serum (HS), were aspirated 2-5 h after plating to remove unattached cells and debris, and replaced with MEM containing 10% fetal bovine serum (FBS). Medium was replaced with MEM/10% FBS every 3-4 days. Astrocytes were kept at 37° C. in a humidified 5% CO₂/95% air incubator for 9-15 days, by which time the cultures were confluent and could be used for experiments. Most of the cells in this preparation (approximately 85% of cultured cells) were immunopositive for glial fibrilary acidic protein (GFAP), the astrocyte-specific intermediate filament protein, and had the characteristics of flat type 1-like astrocytes. The only other cells we were able to identify immunologically in this preparation were microglia (approximately 5% of cultured cells were immunopositive for CD-45). No neurons were detected using neurofilament-specific antibodies. Many of the remaining cells exhibited morphology reminiscent of radial glia that have not yet assumed the genetic program of mature astrocytes [Laywell, E. D., Rakic, P., Kukekov, V. G., Holland, E. C. and Steindler, D. A., Identification of a multipotent astrocytic stem cell in the immature and adult mouse brain, Proc. Natl. Acad. Sci., 97 (2000) 13883-13888].

Drug Preparation

Mc-PAF (Cayman Chemical, Ann Arbor, Mich.) was dissolved in ethanol at a stock concentration of 10 mM. Indomethacin, piroxicam, NS-398 (Biomol; Plymouth Meeting, Mass.), and SC-560 (Cayman Chemical) were dissolved in 45% hydroxy-β-cyclodextrin (HBC; Sigma, St. Louis, Mo.). Cells were serum-deprived for 24 hrs prior to experimental treatments to induce quiescence. Where treatment with inhibitors is indicated, these compounds were added 30 min prior to the addition of mc-PAF.

PGE₂ Assay

Direct assay of the PGE₂ concentration in cell-conditioned medium was used as an index of PGE₂ secretion by primary astrocytes. PGE₂ levels were measured by ELISA according to manufacturer's instructions (Cayman Chemicals, Ann Arbor, Mich.), as described previously [Teather, L. A., Lee, R. K. K. and Wurtman, R. J., Platelet-activating factor increases prostaglandin E2 release from astrocyte-enriched cortical cell cultures, Brain Res., 946 (2002) 87-95].

Data Analysis

Results are derived from at least 3 separate experiments, assayed in duplicate or triplicate (n=6-8). Data are expressed as means +/−SEMs. Statistical analyses were performed using ANOVAs for comparisons between groups, followed by Fischer's PLSD post hoc comparisons by means contrast. p values <0.05 were considered statistically significant.

Results

Effect of mc-PAF on PGE₂ Release

Addition of mc-PAF (0.001 to 1 μM) to the astrocyte-enriched cortical cell cultures resulted in concentration-dependent increases in the release of PGE₂ into the conditioned media (FIG. 8), confirming previous results [Teather, L. A., Lee, R. K. K. and Wurtman, R. J., Platelet-activating factor increases prostaglandin E₂ release from astrocyte-enriched cortical cell cultures, Brain Res., 946 (2002) 87-95]. As these primary astrocytes express both COX-1 and COX-2 according to Western blot analyses (data not shown), we next assessed the involvement of each isozyme in the PAF effect.

Effect of Exposure to Piroxicam Plus SC-650

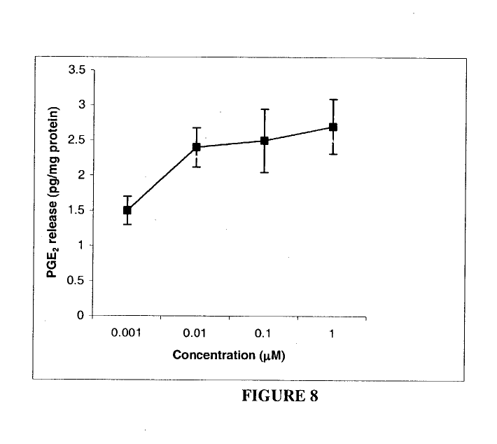

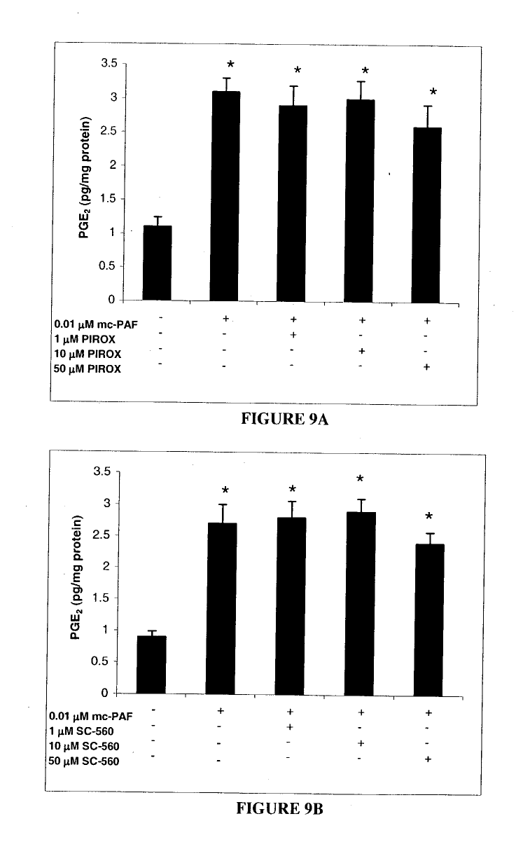

Prior exposure of cells to lower concentrations (1 or 10 μM) of piroxicam (which is considered to be more specific for COX-1 than for COX-2; [Mitchell, J. A., Akarasereenont, P., Thiemermann, C., Flower, R. J. and Vane, J. R., Selectivity of nonsteroidal anti-inflammatory drugs as inhibitors of constituitve and inducible cyclooxygenase, Proc. Natl. Acad. Sci., 90 (1994) 11693-11697]) had no effect on mc-PAF-induced PGE₂ release (FIG. 9A). A higher concentration (50 μM) attenuated some of this PGE₂ release; this effect was not statistically significant. The COX-1 selective inhibitor SC-560 similarly failed to significantly influence mc-PAF-induced PGE₂ release (FIG. 9B). These results suggest that COX-1 activity is not required for PAF-mediated PGE₂ release from astrocytes, even though COX-1 is expressed in these cells.

Effect of Exposure to Indomethacin Plus NS398

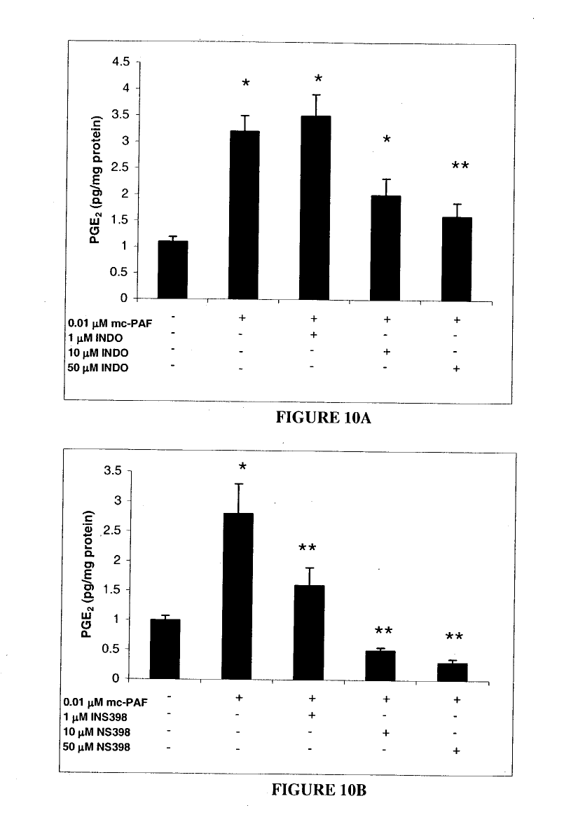

Prior exposure of astrocytes to the non-selective COX inhibitor indomethacin [Meade, E. A., Smith, W. L. and Dewitt, D., Differential inhibition of prostaglandin endoperoxide synthase (cyclooxygenase) isozymes by aspirin and other non-steroidal anti-inflammatory drugs, J. Biol. Chem., 268 (1993) 6610-6614] (1, 10, and 50 μM) attenuated the mc-PAF-induced PGE₂ release in a concentration-dependent manner without affecting basal PGE₂ release (FIG. 10A). The COX-2 selective inhibitor NS-398 [Masferrer, J. L., Zweifel, B. S., Manning, P. T., Hauser, S. D., Leahy, K. M., Smith, W. G., Isakson, P. C. and Seiber, K., Selective inhibition of inducible cyclooxygenase 2 in vivo is antiinflammatory and non-ulceogenic, Proc. Natl. Acad. Sci. (1994) 3228-3232] completely abolished mc-PAF-induced PGE₂ release (FIG. 10B); highest concentrations (10 and 50 μM) also prevented basal PGE₂ release. These results suggest that the COX-2 isozyme is required for PAF-induced PGE₂ release from astrocytes.

Discussion

Cells are thought to have ample basal capacity for COX-catalyzed formation of PGE₂ by expressing either COX-1 or COX-2, or both. However some data suggest that pro-inflammatory stimuli can induce the de novo synthesis of COX-2 protein within minutes in astrocytes [Koyama, Y., Mizobata, T., Yamamoto, N., Hashimoto, H., Matsuda, T. and Baba, A., Endothelians stimulate expression of cyclooxygenase 2 in rat cultured astrocytes, J. Neurochem., 73 (1999) 1004-1011]. The inventors do not believe this to be the case for PAF-induced astrocytic PGE₂ release, for several reasons. First, COX-1 and COX-2 protein levels did not increase within 30 min of mc-PAF stimulation (as assessed by immunocytochemical and Western blot analyses; data not shown). Second, pre-treatment with either a transcription inhibitor (actinomycin D; 5 μg/ml) or a protein translation inhibitor (cyclohexamide; 10 μg/ml) had no effect on mc-PAF-induced PGE₂ release (data not shown). Indeed basal expression of COX-2 appears to be sufficient to sustain the PAF-induced response.

The results of this study show that both PAF-induced and constitutive PGE₂ release are predominantly mediated by COX-2 in astrocytes; and that astrocytes express sufficient basal COX-2 activity to mediate the acute inflammatory response to PAF.

COX-2 is the major enzyme responsible for PG production in developing brain, and astrocytes are an important source of PGE₂ in developing brain [Peri, K. G., Hardy, P., Li, D. Y., Varma, D. R. and Chemtob, S., Prostaglandin G/H synthase-2 is a major contributor of brain prostaglandins in the newborn, J. Biol. Chem., 270 (1995) 24615-24620]. Since the inventors used early post-natal (1-2 days of age) rats to make their cell cultures, it appears that COX-2 can synthesizes astrocytic PGs early in development; indeed PAF-mediated PGE₂ release from astrocytes may have a role in development. It should be kept in mind that cultured astrocytes express elements of a reactive phenotype in culture [McMillian, M. K., Thai, L., Hong, J. S., O'Callaghan, J. P. and Pennypacker, K. R., Brain injury in a dish: A model fro reactive gliosis, TINS, 17 (1994) 138-142], including COX-2 expression [Hirst, W. D., Young, K. A., Newton, R., Allport, V. C., Marriott, D. R. and Wilkin, G. P., Expression of COX-2 by normal and reactive astrocytes in the adult rat central nervous system, Mol. Cell. Neurosci., 13 (1999) 57-68] and may thus provide a model for the activated astrocytes seen in various neurodegenerative and inflammatory-associated disorders. While glial activation can be protective, excess activation can be deleterious [Bolanos, J. P. and Medina, J. M., Induction of nitric oxide synthase inhibits gap junction permeability in cultured rat astrocytes, J Neurochem, 66 (1996) 2091-2099]. In fact, activated astrocytes are neurotoxic in culture systems [Chao, C. C., Hu, S., Sheng, W. S., Bu, D., Bukrinsky, M. I. and Peterson, P., Cytokine-stimulated astrocytes damage human neurons via a nitric oxide mechanisms, Glia, 16 (1996) 276-284] and may be involved in neurodegeneration in vivo [Bolanos, J. P. and Medina, J. M., Induction of nitric oxide synthase inhibits gap junction permeability in cultured rat astrocytes, J Neurochem, 66 (1996) 2091-2099]. Moreover, PGE₂ release has been shown to induce neuronal degeneration [Prasad, K. N., Hovland, A. R., La Rosa, F. G. and Hovland, P. G., Prostaglandins as putative neurotoxins in Alzheimer's disease, Proc. Soc. Exp. Biol., 219 (1998) 120-125]. Our findings suggest that PAF may have a significant role in the inflammatory-immune function of astrocytes by affecting COX-2-mediated PGE₂ release, and could ultimately have a role in inflammatory-immune-associated diseases.

Summary

The phospholipid mediator platelet-activating factor (PAF), and its non-hydrolyzable analog methylcarbamyl-PAF (mc-PAF) increase prostaglandin E₂ (PGE₂) release from astrocyte-enriched cortical cell cultures. Cyclooxygenase (COX) enzymes—of which there are two known isoforms convert arachidonic acid (AA) to prostaglandin (PG) H₂ (PGH₂), which is further metabolized to various PGs, including PGE₂. COX-1 is generally considered to contribute to cell homeostasis, whereas COX-2 is thought to mediate inflammatory/immune PG formation. In this study we examined the involvement of the COX isoforms in PAF-induced PGE₂ release. Treatment of cells with the non-specific COX inhibitor indomethacin, or the specific COX-2 inhibitor NS-398, prior to mc-PAF stimulation completely blocked the PAF-induced release of PGE₂; treatment with more selective COX-1 inhibitors (i.e. piroxicam and SC-560) failed to significantly do so. These data suggest that COX-2 is responsible for PAF-mediated PGE₂ release in primary astrocytes.

Various publications have been referred to throughout this application. The disclosures of these publications in their entireties are hereby incorporated by reference into this application to more fully describe the state of the art to which this invention pertains.

Equivalents

The above examples have been depicted solely for the purpose of exemplification and are not intended to restrict the scope or embodiments of the invention. Other embodiments not specifically described should be apparent to those of ordinary skill in the art. Such other embodiments are considered to fall, nevertheless, within the scope and spirit of the present invention. Thus, the invention is properly limited solely by the claims that follow.

What is claimed is:

1. A method of treating a subject suffering from pain comprising: blocking receptors for platelet-activating factor.

2. The method of claim 1, wherein said blocking is achieved by administering a pharmaceutical composition, and wherein said pharmaceutical composition comprises a pharmaceutically acceptable carrier and a therapeutically effective amount of a benzodiazapine, a tetrahydrofuran, or a derivative thereof, either alone or in combination.

3. The method of claim 1, wherein said blocking is achieved by administering a pharmaceutical composition, and wherein said pharmaceutical composition comprises a pharmaceutically acceptable carrier and a therapeutically effective amount of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, tetrahydrocannabinol-7-oic acid, or a derivative thereof, either alone or in combination.

4. The method of claim 1, wherein said blocking is achieved by administering a pharmaceutical composition, and wherein said pharmaceutical composition comprises a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S,3R,4R)-tetrahydro-2-(3,4-dimethoxyphenyl)-4-(3,4-dimethoxybenzoyl)-3-(hydroxymethyl)furan,
(2S,3R,4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof.

5. The method of claim 1, wherein said blocking is achieved by administering a nutritional supplement, and wherein said nutritional supplement comprises Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C. aureofulvum, C. pubescens, Ardisia elliptica, Goniothalamus malayanus, Kopsia flavida, Momordica charantia, Piper aduncem, Drymis winteri, or derivatives or constitutents thereof, either alone or in combination.

6. The method of claim 1, wherein said blocking is achieved by administering a nutritional supplement, and wherein said nutritional supplement comprises Gingko biloba, or derivatives or constitutents thereof.

7. A method of treating a subject suffering from inflammation comprising: blocking one or more receptors for platelet-activating factor.

8. The method of claim 7, wherein said blocking is achieved by administering a pharmaceutical composition, and wherein said pharmaceutical composition comprises a pharmaceutically acceptable carrier and a therapeutically effective amount of a benzodiazapine, a tetrahydrofuran, or a derivative thereof, either alone or in combination.

9. The method of claim 7, wherein said blocking is achieved by administering a pharmaceutical composition, and wherein said pharmaceutical composition comprises a pharmaceutically acceptable carrier and a therapeutically effective amount of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, tetrahydrocannabinol-7-oic acid, or a derivative thereof, either alone or in combination.

10. The method of claim 7, wherein said blocking is achieved by administering a pharmaceutical composition, and wherein said pharmaceutical composition comprises a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S,3R,4R)-tetrahydro-2-(3,4-dimethoxyphenyl)-4-(3,4-dimethoxybenzoyl)-3-(hydroxymethyl)furan,
(2S,3R,4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

11. The method of claim 7, wherein said blocking is achieved by administering a nutritional supplement, and wherein said nutritional supplement comprises Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C. aureofulvum, C. pubescens, Ardisia elliptica, Goniothalamus malayanus, Kopsia flavida, Momordica charantia, Piper aduncem, Drymis winteri, or derivatives or constitutents thereof, either alone or in combination.

12. The method of claim 7, wherein said blocking is achieved by administering a nutritional supplement, and wherein said nutritional is Gingko biloba, or derivatives or constitutents thereof.

13. A method of treating a subject suffering from pain comprising blocking a site for platelet-activating factor selected from the group consisting of an intracellular PAF receptor binding site and a cell surface PAF receptor.

14. The method of claim 13, comprising blocking an intracellular receptor binding site for platelet-activating factor and blocking a cell surface receptor for platelet-activating factor.

15. The method of claim 13, wherein said blocking is achieved by administering a pharmaceutical composition, and wherein said pharmaceutical composition comprises a pharmaceutically acceptable carrier and a therapeutically effective amount of a benzodiazapine, a tetrahydrofuran, or a derivative thereof, either alone or in combination.

16. The method of claim 13, wherein said blocking is achieved by administering a pharmaceutical composition, and wherein said pharmaceutical composition comprises a pharmaceutically acceptable carrier and a therapeutically effective amount of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, tetrahydrocannabinol-7-oic acid, or a derivative thereof, either alone or in combination.

17. The method of claim 13, wherein said blocking is achieved by administering a pharmaceutical composition, and wherein said pharmaceutical composition comprises a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S,3R,4R)-tetrahydro-2-(3,4-dimethoxyphenyl)-4-(3,4-dimethoxybenzoyl)-3-(hydroxymethyl)furan,
(2S,3R,4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof either alone or in combination.

18. The method of claim 13, wherein said blocking is achieved by administering a nutritional supplement, and wherein said nutritional supplement comprises Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C. aureofulvum, C. pubescens, Ardisia elliptica, Goniothalamus malayanus, Kopsia flavida, Momordica charantia, Piper aduncem, Drymis winteri, or derivatives or constituents thereof, either alone or in combination.

19. The method of claim 13, wherein said blocking is achieved by administering a nutritional supplement, and wherein said nutritional supplement comprises Gingko biloba, or derivatives or constituents thereof.

20. A method of treating a subject suffering from inflammation comprising: blocking an intracellular receptor binding site for platelet-activating factor.

21. The method of claim 20, wherein said inflammation comprises sepsis.

22. The method of claim 20, wherein said blocking is achieved by administering a pharmaceutical composition, and wherein said pharmaceutical composition comprises a pharmaceutically acceptable carrier and a therapeutically effective amount of a benzodiazapine, a tetrahydrofuran, or a derivative thereof.

23. The method of claim 20, wherein said blocking is achieved by administering a pharmaceutical composition, and wherein said pharmaceutical composition comprises a pharmaceutically acceptable carrier and a therapeutically effective amount of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, tetrahydrocannabinol-7-oic acid, or a derivative thereof, either alone or in combination.

24. The method of claim 20, wherein said blocking is achieved by administering a pharmaceutical composition, and wherein said pharmaceutical composition comprises a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S,3R,4R)-tetrahydro-2-(3,4-dimethoxyphenyl)-4-(3,4-dimethoxybenzoyl)-3-(hydroxymethyl)furan,
(2S,3R,4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

25. The method of claim 20, wherein said blocking is achieved by administering a nutritional supplement, and wherein said nutritional supplement comprises Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C. aureofulvum, C. pubescens, Ardisia elliptica, Goniothalamus malayanus, Kopsia flavida, Momordica charantia, Piper aduncem, Drymis winteri, or derivatives or constituents thereof, either alone or in combination.

26. The method of claim 20, wherein said blocking is achieved by administering a nutritional supplement, and wherein said nutritional comprises Gingko biloba, or derivatives or constitutents thereof.

27. A method of inhibiting contraction of the uterus in a subject comprising: blocking receptors for platelet-activating factor.

28. The method of claim 27, wherein contraction inhibition inhibits pain or cramps mediated by premenstrual syndrome, inhibits pain or cramps mediated by menses, inhibits spontaneous miscarriage, inhibits pain or cramps mediated by perimenopausal period, inhibits pain mediated by childbirth or that pain immediately following childbirth, or inhibits Braxton Hicks contractions.

29. The method of claim 27, wherein said blocking is achieved by administering a pharmaceutical composition, and wherein said pharmaceutical composition comprises a pharmaceutically acceptable carrier and a therapeutically effective amount of a benzodiazapine, a tetrahydrofuran, or a derivative thereof, either alone or in combination.

30. The method of claim 27, wherein said blocking is achieved by administering a pharmaceutical composition, and wherein said pharmaceutical composition comprises a pharmaceutically acceptable carrier and a therapeutically effective amount of BN 52021, BN 50730, WEB 286, CV 6209, CV 3988, trans-2,5-Bis(3,4,5-trimethoxypenyl)-1,3-dioxolane, 1-O-hexadecyl-2-O-acetyl-sn-glycero-3-phospho(N,N,N-trimethyl) hexanolamine, octylonium bromide, PCA-4248, tetrahydrocannabinol-7-oic acid, or a derivative thereof, either alone or in combination.

31. The method of claim 27, wherein said blocking is achieved by administering a pharmaceutical composition, and wherein said pharmaceutical composition comprises a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S,3R,4R)-tetrahydro-2-(3,4-dimethoxyphenyl)-4-(3,4-dimethoxybenzoyl)-3-(hydroxymethyl)furan,
(2S,3R,4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

32. The method of claim 27, wherein said blocking is achieved by administering a nutritional supplement, and wherein said nutritional comprises Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C. aureofulvum, C. pubescens, Ardisia elliptica, Goniothalamus malayanus, Kopsia flavida, Momordica charantia, Piper aduncem, Drymis winteri, or derivatives or constituents thereof either alone or in combination.

33. The method of claim 27, wherein said blocking is achieved by administering a nutritional supplement, and wherein said nutritional comprises Gingko biloba, or derivatives or constituents thereof.

34. A method of inhibiting proliferation of tumor cells in a subject in need thereof comprising: blocking receptors for platelet-activating factor.

35. The method of claim 34, wherein said blocking is achieved by administering a pharmaceutical composition, and wherein said pharmaceutical composition comprises a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S,3R,4R)-tetrahydro-2-(3,4-dimethoxyphenyl)-4-(3,4-dimethoxybenzoyl)-3-(hydroxymethyl)furan,
(2S,3R,4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

36. The method of claim 34, wherein said blocking is achieved by administering a nutritional supplement, and wherein said nutritional is Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C. aureofulvum, C. pubescens, Ardisia elliptica, Goniothalamus malayanus, Kopsia flavida, Momordica charantia, Piper aduncem, Drymis winteri, or derivatives or constituents thereof, either alone or in combination.

37. A method of inhibiting neo-angiogenesis in a subject in need thereof comprising: blocking receptors for platelet-activating factor.

38. The method of claim 37, wherein said blocking is achieved by administering a pharmaceutical composition, and wherein said pharmaceutical composition comprises a pharmaceutically acceptable carrier and a therapeutically effective amount of:
trans-2-(3-methoxy-5-methylsulfonyl-4-propoxyphenyl)-5(3,4,5-trimethoxyphenyl)tetrahydrofuran,
(2S,3R,4R)-tetrahydro-2-(3,4-dimethoxyphenyl)-4-(3,4-dimethoxybenzoyl)-3-(hydroxymethyl)furan,
(2S,3R,4R)-tetrahydro-2-(3,4,5-trimethoxyphenyl)-4-(3,4-dimethoxybenzoyl)-3-(hydroxymethyl)furan,
WEB-2086, WEB-2170, Y-24180, BN 50727, BN 50730, BN 50739, E 6123, CV-3988, CV-3938, CV-6209, TCV-309, E5880, SRI 63-441, SM-12502, YM264, ABT-299, SR 27417, UK-74,505, BB-882, WEB 2086, Y-24180, BN 50727, BN 50730, BN 50739, E 6123, or a derivative thereof, either alone or in combination.

39. The method of claim 37, wherein said blocking is achieved by administering a nutritional supplement, and wherein said nutritional comprises Ginkgo biloba, Alphinia galanga, Boesenbergia pandurata, Curcuma aeruginosa, C. domestica, C. ochorrhiza, C. xanthorriza, Aingiber officinale, Z. zerumbet, Cinnamomum altissimum, C. aureofulvum, C. pubescens, Ardisia elliptica, Goniothalamus malayanus, Kopsia flavida, Momordica charantia, Piper aduncem, Drymis winteri, or derivatives or constituents thereof, either alone or in combination.

40. A method of treating acute or chronic pain comprising administering to a subject in need thereof a pharmaceutical composition comprising a PAF antagonist that decreases the release of PAF-mediated increased release of PGE₂ from astrocytes, and a pharmaceutically acceptable carrier or a diluent.

41. The method of claim 40, wherein said decrease is achieved by administering a pharmaceutical composition, and wherein said pharmaceutical composition comprises a pharmaceutically acceptable carrier and a therapeutically effective amount of a COX-2 inhibitor or a derivative thereof.

42. The method of claim 40, wherein said decrease is achieved by administering a pharmaceutical composition, and wherein said pharmaceutical composition comprises a therapeutically effective amount of a COX-2 inhibitor or a derivative thereof, in combination with a COX-1 inhibitor or a derivative thereof, and a pharmaceutically acceptable carrier.

43. A method of treating a subject suffering from inflammation and pain associated with such inflammation comprising decreasing PAF-mediated PGE₂ release in astrocytes, wherein said modulation comprises administering a pharmaceutical composition, wherein said pharmaceutical composition comprises a therapeutically effective amount of a COX-2 inhibitor or a derivative thereof and a pharmaceutically acceptable carrier.

44. The method of claim 43, wherein the therapeutically effective amount of a COX-2-specific inhibitor is sufficient to reduce, ameliorate or completely abolish PAF-mediated PGE₂ release in astrocytes.

45. A method of ameliorating neurodegenerative damage and/or inflammation in vivo in a mammal suffering from a neurodegenerative and/or inflammatory-associated disorder comprising administering a pharmaceutical composition, wherein said pharmaceutical composition comprises a pharmaceutically acceptable carrier and a therapeutically effective amount of a COX-2 inhibitor or a derivative thereof 46. The method of claim 45, wherein the therapeutically effective amount of a COX-2-specific inhibitor is sufficient to reduce, ameliorate or completely abolish PAF-mediated PGE₂ release in astrocytes.
